(19) United States
(12) Patent Application Publication
(10) Pub. No.: US 2007/0287666 A1
(43) Pub. Date: Dec. 13, 2007

(54) Modulator of gamma-secretase

(76) Inventors: Paul E. Fraser, Toronto (CA); Peter H. St. George-Hyslop, Toronto (CA); Gerold Schmitt-Ulms, Toronto (CA); David Westaway, Edmonton (CA)

Correspondence Address: MCCARTHY TETRAULT LLP, BOX 48, SUITE 4700,, 66WELLINGTON STREET WEST, TORONTO, ON, M5K 1E6, CA

(21) Appl. No.: 11/723,708

(22) Filed: Mar. 21, 2007

Related U.S. Application Data
(60) Provisional application No. 60/783,832, filed on Mar. 21, 2006.

(51) Int. Cl. G01N 33/566; A61K 38/17; A61P 25/28; C12N 5/08

(52) U.S. Cl. 514/12; 435/375; 435/7.2

(57) ABSTRACT

The invention relates to modulators of γ-secretase and to methods and uses related thereto. In one embodiment the modulators do not modulate ε-secretase activity. In another embodiment the invention relates to presenilin complex component. In one embodiment the presenilin component is TMP21.

### RELATED APPLICATIONS

This application claims priority from U.S. Provisional Patent Application No. 60/783,832, filed Mar. 21, 2006, entitled “Modulator of Gamma-Secretase”. The entity of which is herein incorporated by reference.

### FIELD OF INVENTIONS

The invention relates to modulators of γ-secretase and to methods and uses related thereto. In one embodiment the modulators do not modulate ε-secretase activity. In another embodiment the invention relates to presenilin complex component. In one embodiment the presenilin component is TMP21.

### BACKGROUND OF THE INVENTION

The presenilin proteins (PS1 and PS2) (1,2) and their interacting partners nicastrin (3), aph-1 (4,5) and pen-2 (5) form a series of high-molecular-mass, membrane-bound protein complexes (6-8) that are necessary for γ-secretase and ε-secretase cleavage of selected type 1 transmembrane proteins, including the amyloid precursor protein (9), Notch (10) and cadherins (11). These transmembrane proteins have been associated with a number of conditions. For instance, amyloid Aβ related conditions, such as Aβ accumulation including Alzheimer's Disease, senile or Amyloid Angiopathy and Inclusion Body Myositis. Modest cleavage activity can be generated by reconstituting these four proteins in yeast and Spodoptera frugiperda (sf9) cells (12-14). However, there is a need to determine how the activity of the presenilin complexes is modulated in terms of substrate specificity and/or relative activities at the γ and ε sites. There is a further need to determine whether additional proteins in the presenilin complexes might subsume these putative regulatory functions. The answers to these questions can lead to new treatments for associated conditions.

As would be expected from the involvement of the presenilin complex in multiple signaling pathways, the absence of any of the four previously known components presenilin complexes causes an embryonic lethal phenotype (43,28), with severe disturbances in developmental signaling (e.g. Notch) in many organ systems, but especially the CNS. The same is true even if the knockout is done post-natally (44).

Current “aspartyl protease-inhibitor-like” γ-secretase inhibitor compounds, which have been found by empirical high throughput screens all have (to greater or lesser degrees) inhibitory effects on both γ-secretase activity and ε-secretase activity (45). This has slowed their deployment into clinical trials. Even for predominantly γ-secretase inhibitors, the residual ε-secretase inhibition becomes relevant at high doses or longer exposures.

Current non-steroidal anti-inflammatory drug (NSAIDs) have selective effects on Aβ42 production (but no effect on Aβ40 and Notch function), but the effect size is small and previous clinical trials of NSAIDs in AD have not been impressive (46,47).

BACE1 (β-secretase) inhibition has limitations both because of the unique structure of the BACE1 active site, and because recent studies have revealed that BACE1 inhibition itself causes mild cognitive and other CNS effects (48).

Anti-Aβ vaccines seem to induce clearing of AD pathology, but have a 6% incidence of auto-immune encephalomyelitis. A work-around using the first few residues of Aβ have been proposed (49).

Statins also have weak effects on Aβ production, and appear to act by changing the intracellular trafficking of APP, rather than by a direct and discrete effect on the enzymes involved in Aβ production. Like with NSAIDs, the therapeutic effectiveness of long term statin therapy is unclear.

In light of the current prior art, there is a further need for a presenilin-interacting protein that differentially affects γ- and ε-site cleavage events, as such a protein can have implications in the treatment of a number of conditions.

### SUMMARY OF THE INVENTION

It is herein reported that TMP21, a member of the p24 cargo protein family, is a component of presenilin complexes and differentially regulates γ-secretase cleavage without affecting ε-secretase activity.

The present inventors have shown herein that:
(a) TMP21 acts as an inhibitor of specific aspects of the function of presenilin complexes.
(b) TMP21 specifically inhibits γ-secretase (which generates the neurotoxic Aβ peptide fragment that plays a central role in the pathogenesis of Alzheimer Disease).
(c) TMP21 has no effect on ε-secretase activity, which is necessary for a multitude of physiological pathways using signaling and growth factor molecules such as Notch, Irep1, APP, p75, and LRP1.
(d) No other p24 cargo proteins are known to have any effect on γ/ε-secretase activities.

In one embodiment, the invention provides a protein, TMP21, or functional active part, analog or derivative thereof or physiologically acceptable salts thereof to inhibit Aβ42 and Aβ40 production (in one embodiment virtually completely blocking γ-secretase activity), but having no effect on ε-secretase activity.

In another embodiment the invention provides a method for developing and/or screening for:
(a) small molecular mimics of TMP21 or method of screening for or producing same;
(b) agonists of TMP21 that bind to the exposed N- or C-termini of TMP21, and either increase targeting of TMP21 into PS-complexes, or increase the affinity of TMP21 for PS-complexes.

In another embodiment the invention provides a method for deducing the structure of TMP21 (which is a small single spanning transmembrane protein that does not undergo post-translational modifications) more easily than any of the other presenilin complex components (PS1 (1), PS2 (2), nicastrin (3) aph-1(4), pen-2 (5)) that are either multi-spanning transmembrane proteins, highly glycosylated, and/or highly hydrophobic.

In one embodiment, the invention provides the use of recombinant TMP21 in high-throughput mass-spectrometry-based screens (e.g. Optimol™) for compounds binding to TMP21, and the use of those compounds to rapidly re-screen for effects on γ/ε-secretase activities.

In another embodiment, the invention provides a method for screening for TMP21 agonists.

In another embodiment, the invention provides TMP21, analogs, derivatives, physiologically acceptable salts and modulators of same that can be used in the screening for compounds and in the treatment and diagnosis of a number of aforementioned conditions, such as in a disease where Aβ accumulates, namely:
(a) Various forms of Amyloid (Congophilic) Angiopathy (including Senile Amyloid Angiopathy, a common cause of stroke and lobar cerebral hemorrhages in the elderly (11)); and
(b) Inclusion Body Myositis (the most common cause of myopathy in the elderly (12)).

In one embodiment, the invention provides a method for modulating γ-secretase activity, in one embodiment, not ε-secretase activity, in-vitro sample or in-vivo in a subject comprising administering to said sample or subject TMP21 or obvious chemical equivalent thereof. In another embodiment, the invention provides a use of TMP21 or obvious chemical equivalent thereof for modulating γ-secretase activity in-vitro sample or in-vivo in a subject. In one embodiment, the TMP21 is a presenilin complex associated peptide. In one embodiment, the modulating γ-secretase activity is inhibiting γ-secretase activity but not ε-secretase activity. In another aspect, the method is used to decreasing Aβ production.

In another aspect, the invention provides a method for preventing or treating a condition associated with γ-secretase activity but not ε-secretase activity comprising administering to a subject an effective amount of TMP21 or obvious chemical equivalent thereof. In another aspect, TMP21 or obvious chemical equivalent thereof can be used to prevent or treat a a condition associated with γ-secretase activity but not ε-secretase activity, such as an amyloid Aβ-related condition. In one embodiment, the amyloid Aβ-related condition is selected from the group consisting of: Alzheimer's, cerebral amyloid angiopathy, and inclusion body myositis. In another embodiment, the condition is Alzheimer's.

In another embodiment, the invention provides a method for diagnosing a γ-secretase related condition comprising obtaining a biological sample from a subject, such as in one embodiment by way of a lumbar puncture, that is suspected of comprising presenilin complexes and/or, TMP21 and/or an indicator of TMP21 levels, such as Aβ; determining TMP21 levels in said sample, comparing the TMP21 level with control levels from patients with known disease states, diagnosing the subject based on comparing TMP21 levels in said patient to the control levels and rendering a diagnosis based on said comparison with patients of known disease state.

In one embodiment, the TMP21 levels are determined directly, for instance by by assessing levels of nucleotide sequence encoding TMP21 (e.g. RT-PCR) or are determined through binding studies, such as antibody binding studies or other labeling techniques known in the art. In another embodiment, TMP21 levels are determined indirectly by indicators of TMP21 levels, such as, by assessment of γ-secretase activity and or Aβ production.

In one embodiment, control levels in the methods of the invention are based on subjects or samples with no γ-secretase related condition and TMP21 levels that are lower than those of the control is indicative of a γ-secretase related condition.

In one embodiment, the γ-secretase related condition is selected from the group consisting of Alzheimer's, cerebral amyloid angiopathy, and inclusion body myositis, or in another embodiment Alzheimer's.

The invention further provides a method of monitoring the disease state of a subject with a γ-secretase related condition comprising monitoring levels of TMP21 activity in biologicial samples obtained from a subject over time, wherein a decrease in TMP21 levels over time is indicative of a worsening of or progression of the condition, while maintaining or increasing TMP21 levels over time is indicative of non-progression of the disease state. In one embodiment for monitoring disease progression, TMP21 is being used in the treatment of the condition and the method is used to monitor the effectiveness of the treatment.

In another embodiment, the invention provides a method of identifying modulators of γ-secretase activity that are not modulators of ε-secretase activity comprising incubating γ-secretase or a biologically active source therefore with APP substrate under conditions wherein the secretase would cleave the APP to form Aβ, monitoring Aβ production in both the presence and absence (control) of a potential modulator, wherein a change in Aβ production as compared to the control is indicative of a modulator. In one embodiment, the method further comprises monitoring levels ε-secretase activity, and selecting modulators that have no change in ε-secretase activity as compared to a control. In a further embodiment, the ε-secretase activity is monitored by monitoring levels of intracellular fragments of Notch and/or Cadherin (e.g. NICD or CICD). In a further embodiment, the modulator is an inhibitor of γ-secretase activity and has lower Aβ production levels as compared to a control. In another aspect of the invention the potential modulator is first screened in a TMP21 binding assay and was determined to bind TMP21. control is the presence of TMP21 but no potential modulator and/or the presence of TMP21 plus the potential modulator, and/or the present of TMP21 and a known modulator of TMP21.

In one embodiment, the controls used in the methods of the invention can be those known to a person skilled in the art upon reading this description. In one embodiment, the control is a method for screening for TMP21 modulators that selectively regulate γ secretase comprising: incubating APP with γ-secretase under conditions that would result in Aβ production, exposing said APP, gamma-secretase sample to a potential inhibitor of gamma secretase activity, monitoring the effect of said activity on Aβ production as compared to a control. In another aspect, method is done in the presence and absence of TMP21 and any change in TMP21 activity in the presence of the potential modulator as compared to no potential modulator is indicative that the potential modulator is a modulator of TMP21. In another aspect the potential modulator is first screened in a TMP21 binding assay and was determined to bind TMP21.

In a further embodiment, the invention provides a pharmaceutical composition comprising TMP21, a pharmaceutically acceptable salt thereof or obvious chemical equivalent thereof and a pharmaceutically acceptable carrier.

Additional aspects and advantages of the present invention will be apparent in view of the description which follows. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE FIGURES

The invention will now be described in relation to the drawings, in which:

FIGS. 1-9 are in this application on 21 number of figure pages and are described in the Detailed Description of the Invention and Examples herein.

FIG. 1 is as described in Example 1. FIG. 1A is a western blot study of the immunoprecipitated gamma-secretase complex indicating the presence of the major components (PS1, NCT, APH-1, PEN-2) and their interaction/co-isolation with TMP21.

FIG. 1B is a similar western blot examination of the complex probing for the related p24a indicating that this protein is not bound to the gamma-secretase components.

FIG. 1C is an immunoprecitation study from different cells (HEK293 and SHSY-5Y) and tissues (mouse brain extracts) which validates the binding of TMP21 to the complex in vivo.

FIG. 1D is a glycerol gradient fractionation demonstrating the distribution of the gamma-secretase complex components and their overlap with TMP21.

FIG. 1E is a Blue Native 2D gel electrophoresis study in normal and complex deficient fibroblasts.

FIG. 2 is as described in Example 2. FIG. 2A is a western blot analysis of complex components and APP-related substrates in normal and TMP21 knockdown cells and their effects on generation of the amyloid-beta peptides using different model systems and assays.

FIG. 2B is a quantification of changes in Abeta levels following suppression of TMP21.

FIG. 2C demonstrates the effects of different siRNA oligonucleotides to validate the specificity of the observed suppression of TMP21 and not the related p24a.

FIG. 2D is a schematic representation of the two proposed pools of cellular TMP21 that is bound to either the cargo protein complex or PS1-gamma secretase.

FIG. 2E is a biotinylation study that demonstrates the trafficking of TMP21 and nicastrin to the cell surface (GM130 is a negative control).

FIG. 3 is as described in Example 3. FIG. 3A indicates an in vitro rescue study where the addition of exogenous recombinant TMP21 to an isolated gamma-complex reduces the observed increase in Abeta peptide.

FIG. 3B is a quantitative representation of the data shown in FIG. 3A.

FIG. 3C is an evaluation of Abeta levels following over-expression of TMP21 and indicates no effect of elevated TMP21 levels.

FIG. 3D is a control siRNA study that demonstrates the specificity of the TMP21 siRNA as compared to an unrelated oligonucleotide.

FIG. 4 is as described in Example 4. FIG. 4A is a western blot study of the effects of TMP21 suppression on Abeta and AICD generation.

FIG. 4B is a pulse-chase study demonstrating the lact of effect of TMP21 suppression on epsilon-cleavage of Notch.

FIG. 4C is a pulse-chase study demonstrating the lact of effect of TMP21 suppression on epsilon-cleavage of cadherin.

FIG. 5 is as described in Example 5. FIG. 5A is a western blot analysis for Abeta and AICD changes following siRNA knockdown of p24a demonstrating no effect of this related cargo protein.

FIG. 5B is a quantification of the Abeta changes following p24a suppression.

FIG. 6 is as described in Example 6. FIG. 6 is a western blot study in normal and PS1/2 knockout cells that have also been treated with the gamma-secretase inhibitor (Compound E) to demonstrate that TMP21 is not a substrate of the complex.

FIG. 7 is as described in Example 7. FIG. 7A is a pulse-chase study in normal, TMP21 suppressed and siRNA control cells that demonstrates no changes in the level of APP substrate.

FIG. 7B is a western blot study to examine the maturation and trafficking of the gamma-secretase complex component (nicastrin), the APP substrate and the beta-secretase protease (BACE).

FIG. 8 is as described in Example 8. FIG. 8 is a western blot analysis of a number of different TMP21 siRNA oligonucleotides indicating that all had a specific effect on protein expression.

FIG. 9 is as described in Example 9, A) A ‘clustal W’ alignment of human TMP21, yeast Erv25p and yeast Emp24p. TMP21 shares 58% sequence identity with Erv25p. All of these proteins are members of the ‘p24 family’ of proteins that play a role in vesicle trafficking between the E.R. and Golgi. Emp24p and Erv25p are two members of a heteromeric protein complex that also includes Erp2p and Erp1p. A yellow box has been placed around the first 15 amino acids of TMP21 that do not align with Erv25 or Emp24. B) Graph showing the effect of expressing TMP21 in conjunction with the other four mammalian γ-secretase components on lacZ reporter activity in ethanol containing medium. The suppression of γ-secretase activity by co-expression of TMP21 parallels its behavior in mammalian systems in both the PMY1 (wt) and the DEY1 (V7171) yeast strains. C) Western blot using anti-FLAG (M2) antibody to detect TMP21-Flag co-expression. PEN-Flag expression is also detected by the same antibody. The asterisk indicates a cross-reacting band.

### DETAILED DESCRIPTION OF THE INVENTION

To isolate additional presenilin complex components an affinity-purified polyclonal antibody (A4) directed against the amino terminus of PS1 was used to immunoprecipitate PS1 complexes from CHAPSO-solubilized membranes of wild-type blastocyst-derived cells expressing PS1 and PS2, and from similar membranes of PS1^{-/-}/PS2^{-/-} double-knockout blastocyst-derived cells (which served as a negative control). The complexes immunopurified from wild-type cells had a mass of at least 650 kDa and possessed γ-secretase enzymatic activity (6). The immunoprecipitates from both wild-type and PS1/PS2 double-knockout cells were subjected to in-solution tryptic disgestion, and the protein components were then identified by mass spectrometry.

All four known constituents of the PS1 complex (nicastrin (3), aph-1 (4,5) pen-2 (5) and PS1 (1)) were present only in the co-precipitates from wild-type cells. Among the few other proteins that were uniquely present only in the immunoprecipitates from wild-type presenilin-expressing cells but not in those from the PS1^{-/-}/PS2^{-/-} cells, the strongest identification was made for TMP21 (accession number Q9D1D4) based on three unique tryptic peptides (LKPLEVELR (SEQ ID NO:1), IPDQLVILDMK (SEQ ID NO:2) and RLEDLSESIVNDFAYMK (SEQ ID NO:3)) covering 17.5% of the full protein length. TMP21, a 219-amino-acid type 1 transmembrane protein, is a member of the p24 cargo-protein family (15) that is involved in protein transport and quality control in the endoplasmic reticulum and Golgi (16). TMP21 protein also resides at the plasma membrane (15) (which is one of the principal subcellular locations for γ- and ε-cleavage of many substrates). The gene encoding TMP21 is located on chromosome 14 in a highly conserved cluster of genes that maps close to PS1 itself (1,17). As might be predicted, recent bioinformatic analyses reveal that TMP21, PS1 and amyloid precursor protein (APP) display dynamic patterns of co-transcription (18).

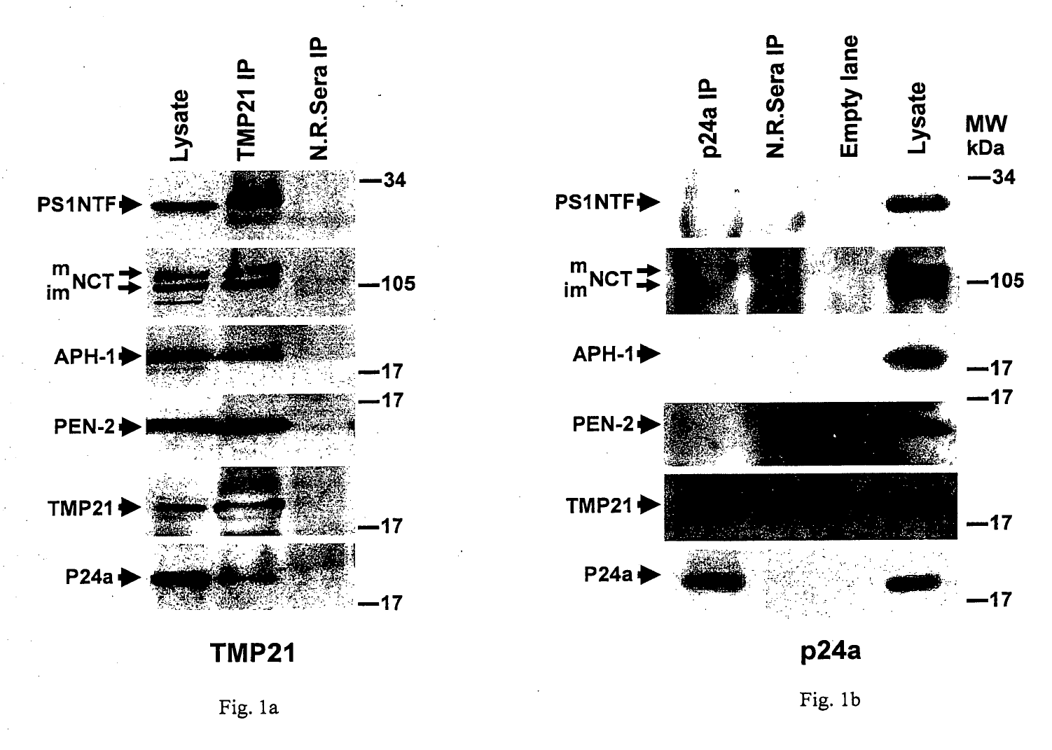

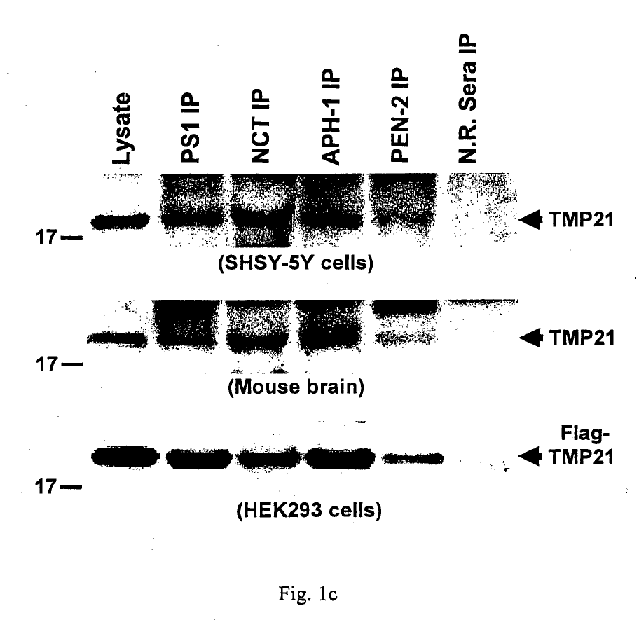

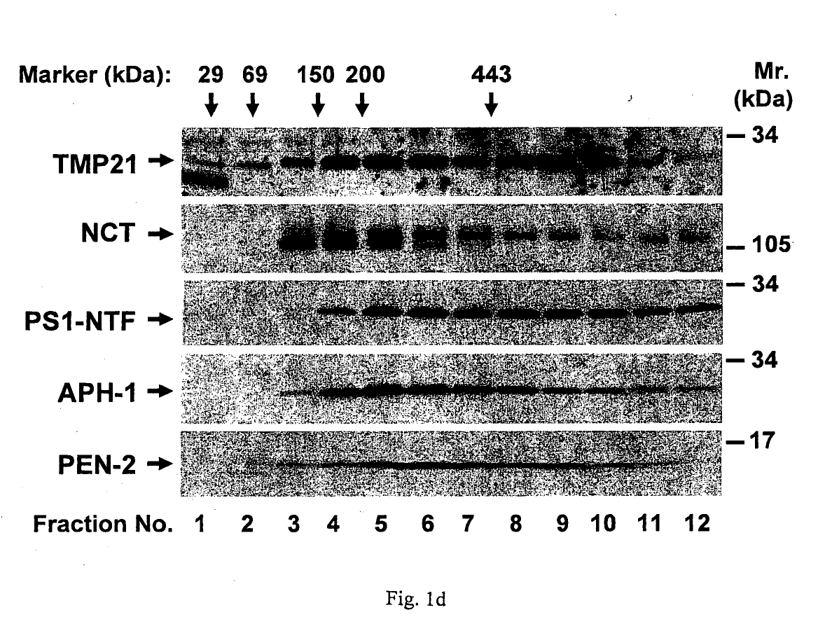

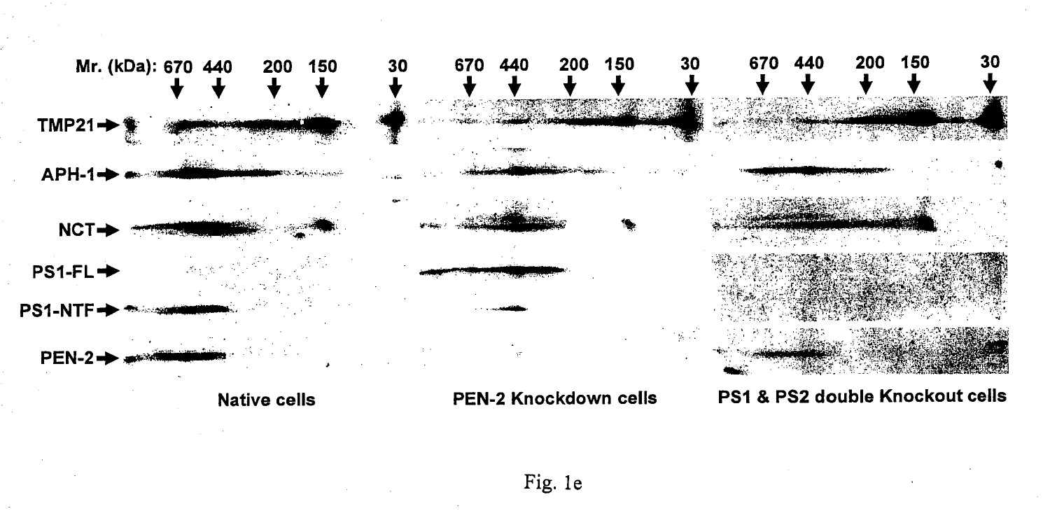

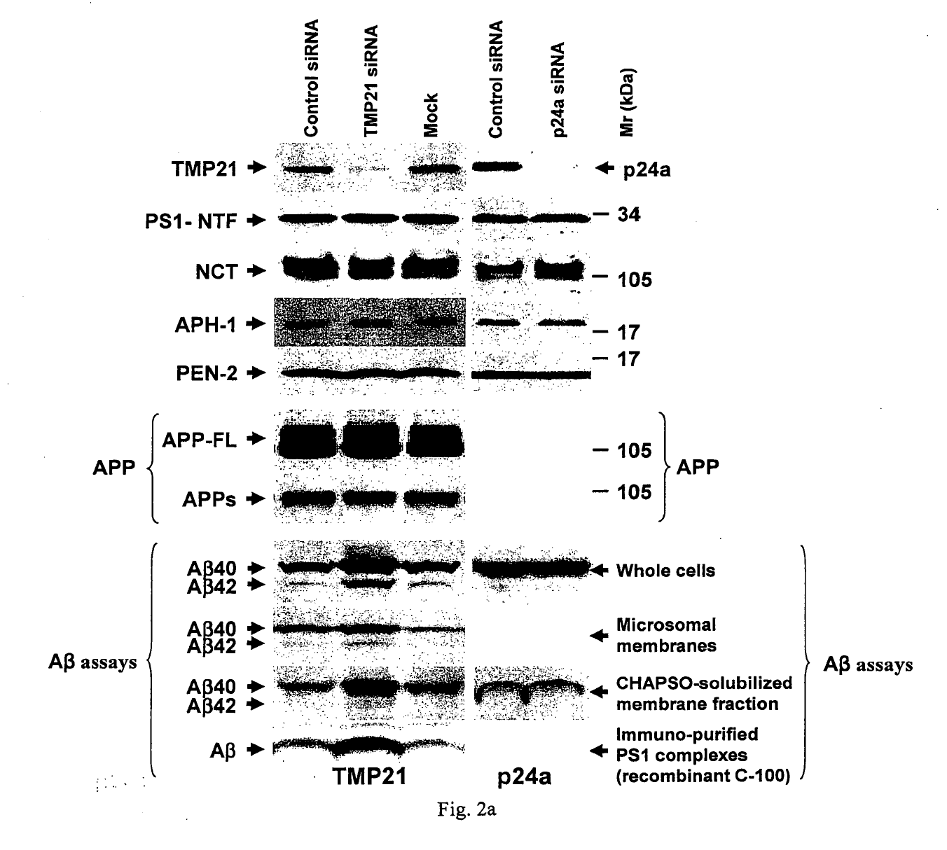

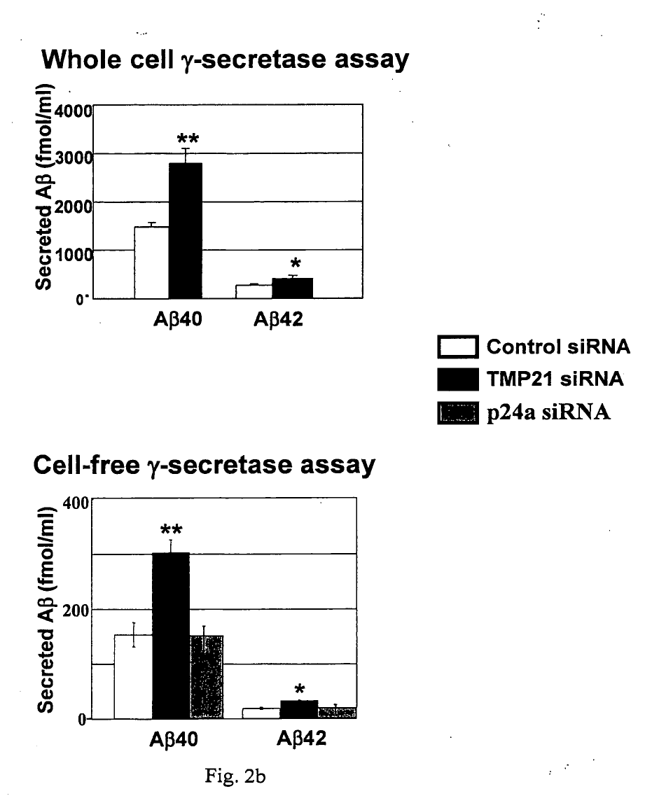

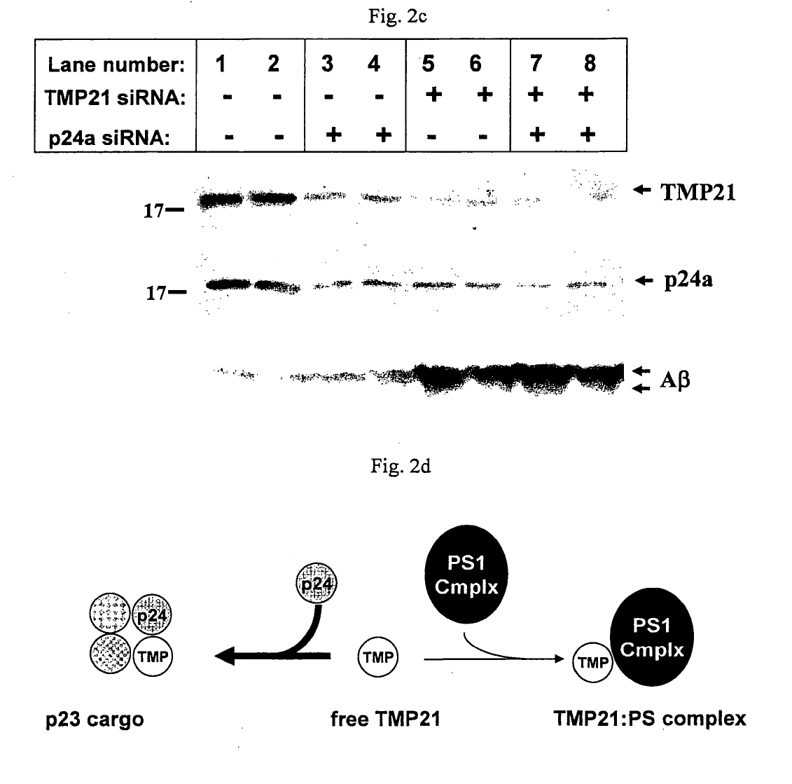

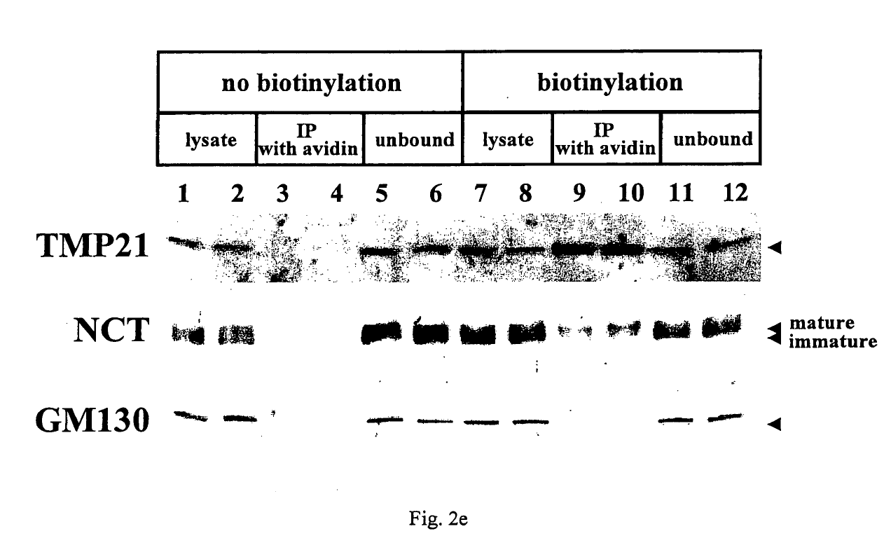

The authenticity of the interaction between TMP21 and the presenilin complex was confirmed by showing that endogenous TMP21 from mouse brain, neuron-like SHSY-5Y cells and human embryonic kidney (HEK-293) cells could be co-immunoprecipitated with endogenous nicastrin, aph-1, pen-2 and PS1 (FIG. 1a), and that it had an overlapping size distribution with presenilin complex components in high-molecular-mass (more than 650-kDa) fractions on glycerol velocity gradients and on two-dimensional Blue Native gel chromatography (FIG. 1d, e). Furthermore, in the absence of PS1 and PS2, or in the absence of pen-2, TMP21 was destabilized from the complexes with a molecular mass of more than 650 kDa, co-localizing predominantly with an approximately 150-kDa nicastrin-aph-1 complex and with an approximately 440-kDa complex containing the remaining presenilin complex components (FIG. 1e). TMP21 could be surface biotinylated, as can nicastrin (FIG. 2e), and co-localized with presenilin complex components in biochemical fractionation and immunofluorescence studies in the endoplasmic reticulum, Golgi and cell surface (not shown). In contrast, p24a, another member of the p24 cargo-protein family with 48% amino-acid sequence similarity to TMP21 (E=7×10⁻⁹), (where E=expected number statistic for high scoring sequence alignment pairs) forms heteromeric complexes with TMP21 in the endoplasmic reticulum and Golgi (15,16), but does not co-precipitate with presenilin complex components (FIG. 1b).

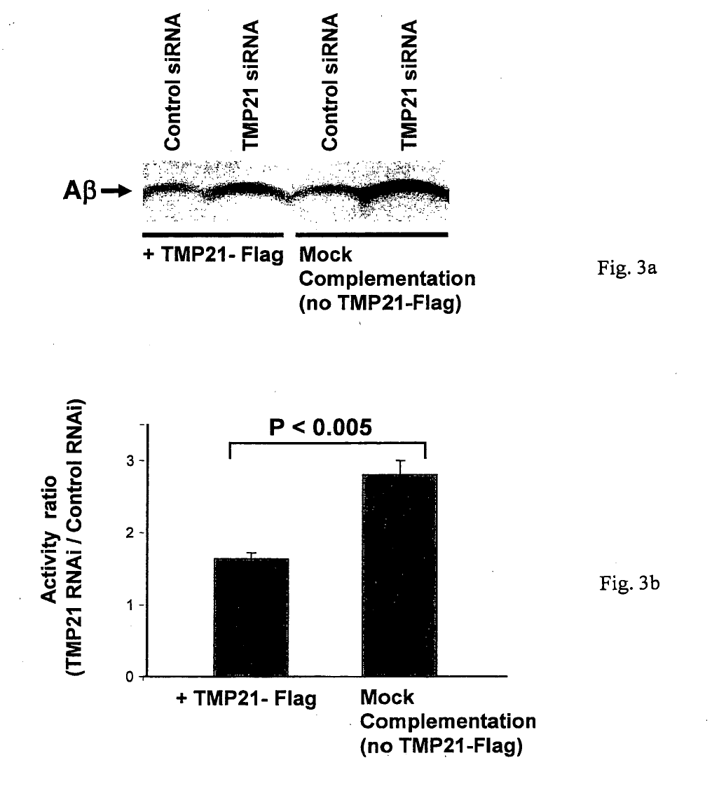

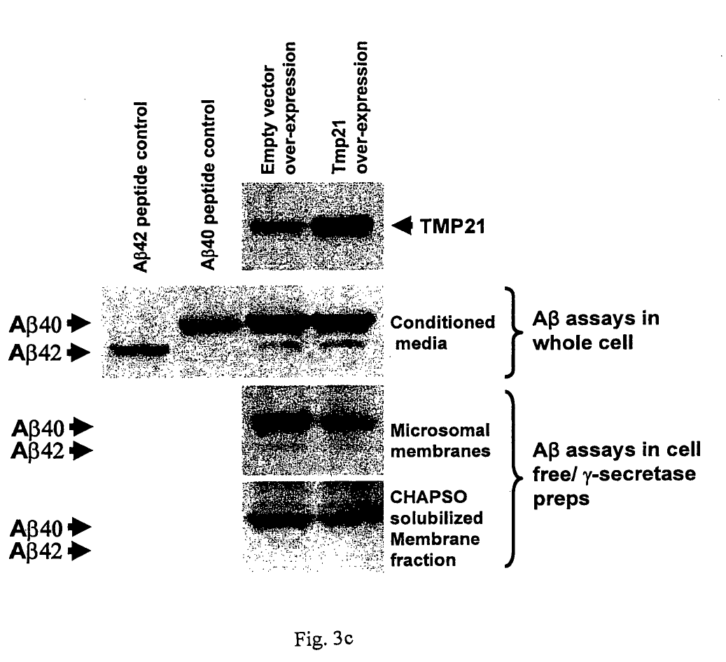

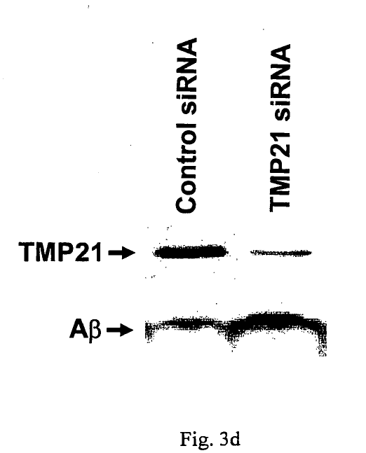

To assess the functional consequences of the interaction between TMP21 and presenilin complexes, we next investigated the effects of modulating expression of TMP21. Transient overexpression of TMP21 had no discernible effect on the abundance of the presenilin complex components, on the abundance of p24 proteins such as p24a, on the subcellular distribution of these proteins (as assessed by both biochemical fractionation and immunocytochemistry data (not shown)), or on Aβ production in either whole HEK-293 cells or in cell-free γ-secretase assays (FIG. 3c).

In contrast, when TMP21 expression was suppressed by small interfering RNAs (siRNAs) there was an increase in the production of both Aβ40 and Aβ42. The increase in Aβ production was observed in whole HEK-293 cells overexpressing APPswedish (Aβ was 189±20.70% (mean±s.e.m.) of control, n=5, P<0.005; FIG. 2a, left panel, and FIG. 2b), in HEK-293 cells overexpressing wild-type APP (216±20.07% of control, n=5, P=0.005; not shown), in native HEK-293 cells expressing endogenous APP (181±17.18% of control, n=5, P=0.005; not shown) and in neuron-like SHSY-5Y cells (219±10%, n=3, P<0.01; FIG. 3d). Similar increases in Aβ production were also observed in cell-free γ-secretase assays of TMP21-deficient presenilin complexes with either endogenous ‘pre-docked’ C100-APP substrate (FIG. 2a, left panel, and FIG. 2b), or with an exogenous recombinant C100-APP substrate (FIG. 2a, left panel, and FIG. 2b). This increase in γ-secretase activity was not accompanied by either changes in the levels of endogenous PS1, nicastrin, aph-1, pen-2, N′O′-glycosylated APP holoprotein or changes in α-secretase or β-secretase activity (the levels of secreted N-terminal APP ectodomain fragments in the conditioned media were unaltered; FIG. 2a, left panel). These results indicate that although TMP21 might be a component of presenilin complexes, it is not essential for the assembly of the presenilin complexes. This result also indicates that TMP21 modulates γ-secretase activity by a method other than by simply altering presenilin complex assembly and stability.

The increase in Aβ secretion after suppression of TMP21 was specific to TMP21. Neither transient overexpression (FIG. 3c) nor siRNA-mediated suppression of p24a (FIG. 2a, right panel, and FIG. 2b) had any effect on either the abundance of the presenilin complex proteins or on Aβ production, regardless of whether whole cells or CHAPSO-solubilized microsomal membranes were used (FIG. 5). However, as expected, p24a suppression did decrease TMP21 levels in whole cells (FIG. 2c). This apparent paradox, in which there is no change in Aβ production when decreases in TMP21 are caused by decreases in p24a but where there are profound changes in Aβ production when TMP21 itself is suppressed, can be explained by the existence of two pools of TMP21 (FIG. 2c, bottom panel). The major pool is stabilized by p24a but has no direct role in Aβ production. In contrast, the second and smaller pool of TMP21 interacts tightly with presenilin complexes and regulates Aβ production. The existence of these two pools of TMP21 is shown by the following experiments. The decrease in p24a-associated TMP21 (through p24a suppression) had no effect on Aβ levels. However, there was a significant increase in Aβ production when the residual ‘non-p24a-associated’ TMP21 in these p24a siRNA-treated cells was subsequently suppressed by TMP21 siRNA. This increase in Aβ production in the double (p24a and TMP21) siRNA-suppressed cells was equivalent to the increase in Aβ production induced by the siRNA suppression of TMP21 alone (FIG. 2c).

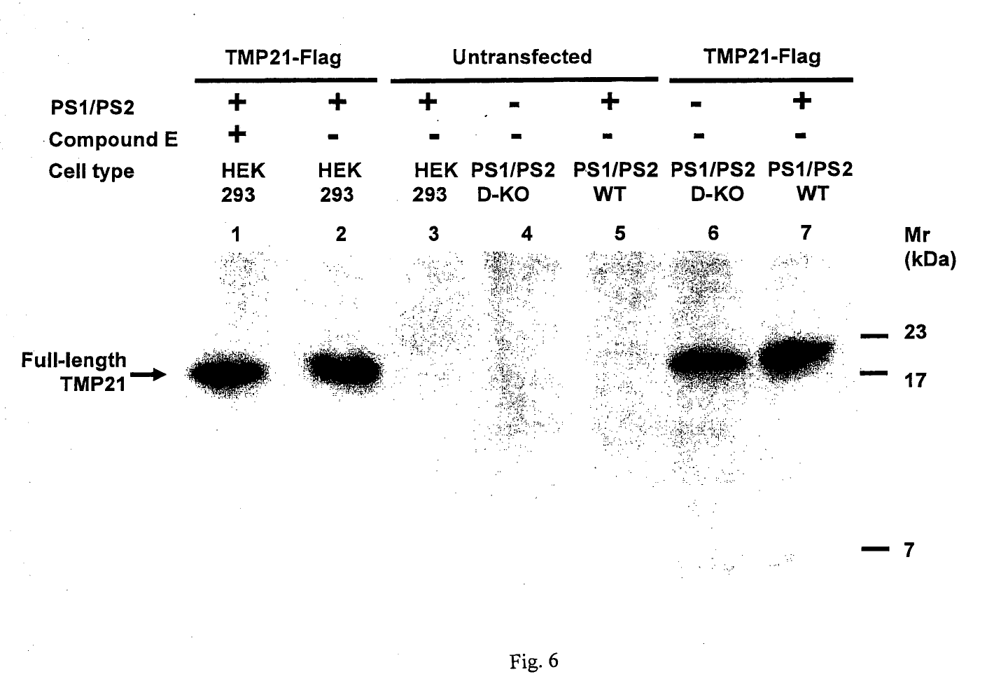

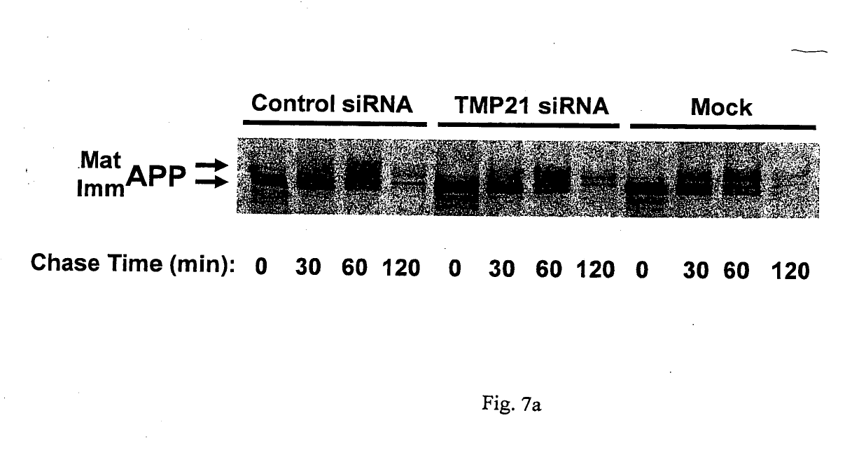

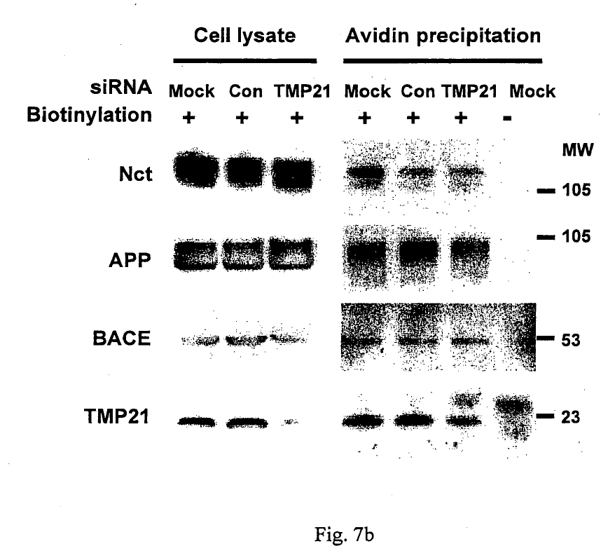

TMP21 could regulate the activity of the presenilin complex through at least three mechanisms. First, it is conceivable that TMP21 might be a competing substrate for presenilin-mediated endoproteolysis. This hypothesis can be excluded by the following facts: no N-terminal secreted products of TMP21 could be detected in conditioned medium (data not shown); no C-terminal TMP21 fragments corresponding to a putative γ-site cleavage product could be detected in cell lysates; and no carboxy-terminal precursors for γ-secretase cleavage of TMP21 equivalent to α- or β-secretase stubs were detectable even in cells treated with potent γ-secretase inhibitors (such as compound E) or in cells deficient in both PS1 and PS2 (FIG. 6). Second, as a member of the p24 cargo protein family, it is conceivable that TMP21 might modulate the maturation and/or subcellular trafficking of the presenilin complex and its substrates. However, pulse-chase and surface biotinylation analyses, after either overexpression or underexpression of TMP21, revealed no detectable changes in the glycosylation, maturation, abundance or temporal patterns of trafficking of the APP substrate or of the presenilin complex components to the cell surface (FIG. 7a, b). Thus, suppression of TMP21 had no effect on the abundance of APP and nicastrin at the cell surface as measured by surface biotinylation (FIG. 7b), on the patterns of glycosylation of APP and nicastrin (FIG. 2a), or on the patterns of endoproteolysis of PS1 (FIG. 2a).

The third potential mechanism is that, in addition to its general role as a p24 cargo protein, TMP21 might also act as a direct modulator of the presenilin complex itself (FIG. 2c). To resolve whether TMP21 had a direct effect on the function of presenilin complexes that was independent of any role that TMP21 might have on trafficking. The effects of adding exogenous Flag-tagged TMP21 to immunopurified, TMP21-deficient presenilin complexes were investigated. These cell-free complementation experiments, in which issues relating to trafficking can be excluded, revealed that Aβ production reverted to normal when exogenous TMP21 was added to the immunopurified TMP21-deficient PS1 complexes (FIG. 3). However, Aβ production remained elevated in the ‘mock-complemented’ TMP21-deficient PS1 complexes (FIG. 3).

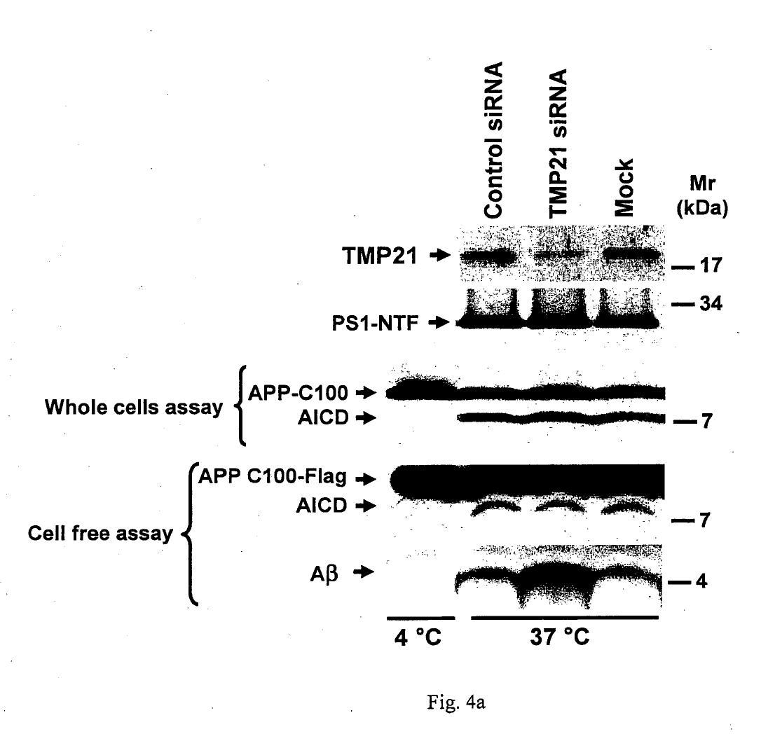

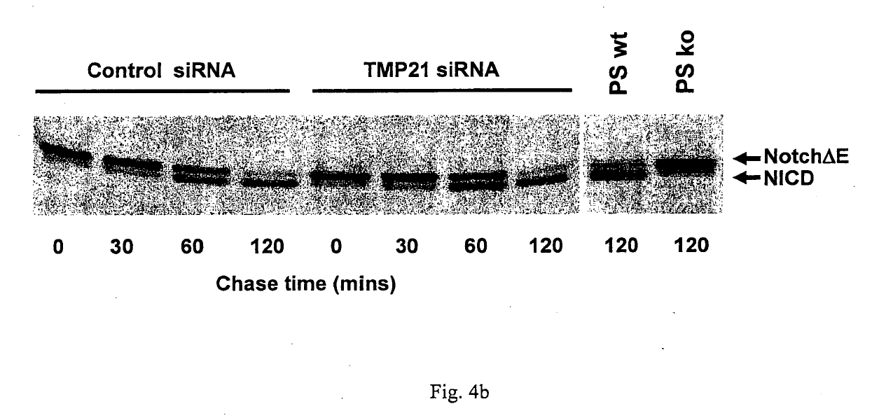

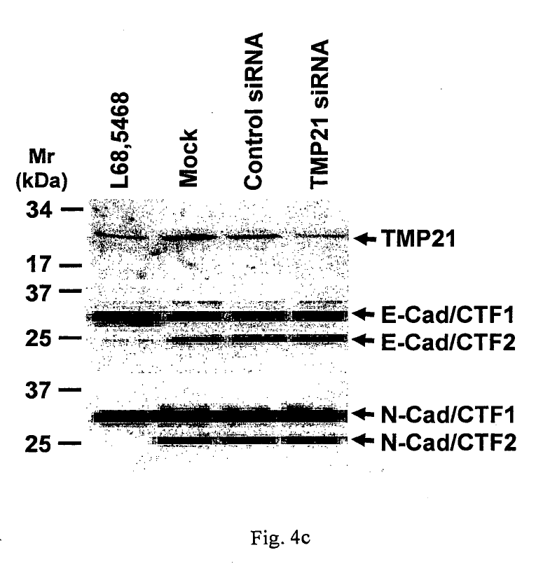

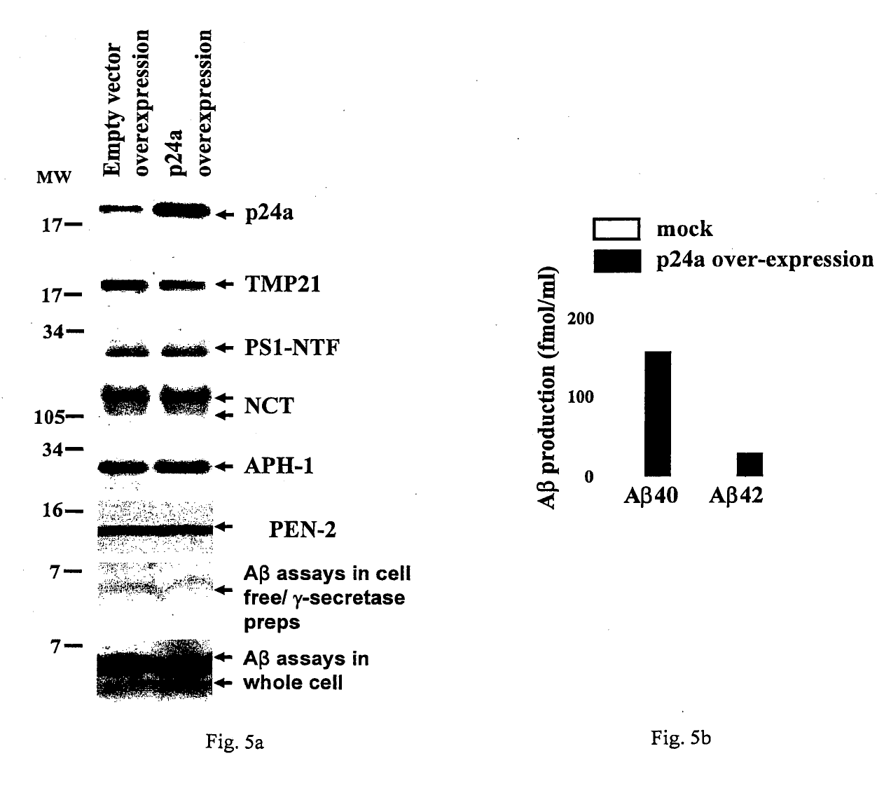

Surprisingly, and in notable contrast to its effect on γ-secretase activity, the suppression of TMP21 by siRNA had no discernible effect on ε-secretase activity as measured by the production of the amyloid intracellular domain (AICD), the Notch intracellular domain (NICD) or the Cadherin intracellular domain (CICD) (FIG. 4). This result was robust regardless of whether ε-secretase activity was assayed in whole cells with endogenous substrate or in purified complexes with a recombinant substrate (FIG. 4a). This result also strongly supports the conclusion that the effect of TMP21 suppression is not due to a simple defect in vesicular trafficking, but is due to a specific effect on one aspect of the function of PS1 complexes. Nevertheless, this result is surprising because previous studies have shown that suppression of the Caenorhabditis elegans homologue of p24a (sel-9) restores the signalling activity of mutant lin-12 or glp-1 proteins by permitting trafficking of the mutant proteins to the cell surface (19). However, this latter effect probably reflects the separate general role of p24 cargo proteins in the quality control of mutant proteins in the secretory pathway (that is, the suppression of sel-9 expression in cells expressing mutant lin-12/glp-1 abrogates the normal quality-control function of sel-9, which would normally inhibit the transport of the mutant lin-12 and glp-1 to the cell surface) (19).

Taken together, the data herein indicate that, in addition to its function in protein transport and quality control within the secretory pathway, TMP21 also has a specific role as a modulator of presenilin-dependent γ-site cleavage. This newly discovered property is highly specific to the γ-site cleavage, and TMP21 does not modulate cleavage at the ε site. Moreover, this effect is restricted to TMP21 and is not a general property of other p24 cargo proteins.

The concept of a multimeric protein complex that releases constitutive inhibitory subunits only in response to specific stimuli is analogous to the NF-κB/IκK complex (20). The results herein indicate TMP21 provides a method of both preventing runaway intramembrane proteolysis and of coupling such a putative activity to other quality-control mechanisms mediated by TMP21 and its p24 cargo protein partners.

The present inventors' observation that γ- and ε-site cleavages are independently regulated strongly indicates that γ- and ε-site secretase cleavages are distinct but related properties of the presenilin complexes and are not simply the reflection of a loosely specified cleavage site by a single enzymatic activity. This conclusion is supported by the previous observations that some presenilin mutations (24) and some γ-secretase inhibitors (25) also have differential effects on γ- and ε-site cleavages. The findings of the present invention will be of use in the design of γ-site-specific inhibitors for the treatment of Alzheimer's disease. For instance, in one embodiment of the invention one can determine whether the binding of small molecules or peptides to the luminal N terminus or to the cytosolic C terminus of TMP21 might regulate the effect of TMP21 on γ-site cleavage. These resulting ligands, or molecular mimics of TMP21 itself, can provide a way to manipulate γ-site cleavage (and thus Aβ production in patients with Alzheimer's disease) therapeutically, without altering ε-site cleavage (which is necessary for many physiological signal transduction mechanisms, including Notch signalling).

### DEFINITIONS

“TMP21” as used herein means a protein or fragment thereof having an amino acid sequence identical to or substantially similar to that disclosed for TMP21 in Genbank Accession Nos. Q9D1D4, NP_{—}006818 and AAD31941. Polypeptides which are “substantially similar” to TMP21 disclosed in these Genbank Accession Nos. may contain conservative amino acid substitutions which do not alter the structure or activity of TMP21. When the diagnostic method of the invention is used to diagnose or monitor a γ-secretase-related condition in a species other than a human, the term “TMP21” used herein includes TMP21 from that species. The term also includes all homologs, naturally occurring allelic variants, isoforms and precursors of TMP21. In general for example, naturally occurring allelic variants of TMP21 will share significant homology (70-90%) to the sequences shown in the aforementioned GenBank Accession Nos. TMP21 fragments are fragments that have the biologically activity of the full length TMP21 with respect to γ-secretase inhibitory activity and lack of ε-secretase modulating activity.

“TMP21 nucleic acids” is meant to include both RNA and DNA encoding TM21 as defined herein with the same structure and activity.

“Obvious chemical equivalent” as used herein as used in reference to TMP21 refers to salts, analogues, derivatives, polymorphs, mutations of TMP21 that have γ-secretase inhibitory activity and lack ε-secretase modulating activity.

“γ-secretase-related condition” as use herein means a medical condition associated with γ-secretase activity or function, or production, or expression, such as amyloid Aβ-related conditions.

“amyloid Aβ-related condition” as used herein means a medical condition that is associated with Aβ production, expression, accumulation or activity, such as Alzheimer Disease, (Alzheimer's) Amyloid Angiopathy (such as Amyloid Congophilic Angiopathy, including Senile Amyloid Angiopathy, a common cause of stroke and lobar cerebral hemorrhages in the elderly), and Inclusion Body Myositis (common cause of myopathy in the elderly).

“Modulator” as used herein means a substance that modulates the activity or expression of another peptide, gene or chemical. It includes, positive and negative modulators and includes substances that can maintain a particular activity or expression level under conditions where up or down regulation of said activity or expression would normally change, if the modulator was not present.

“Presenilin complex associated peptide” as used herein means a peptide associated with the presenilin complex, such as TMP21.

“Subject” as used herein refers to a warm-blooded animal such as a mammal Preferably, “subject” refers to a mammal, most preferably a human.

“Sample”, “biological sample”, and the like mean a material known or suspected of expressing or containing presenilin complex or γ-secretase or APP or Aβ or TMP21 associated with presenilin complex. The sample can be used directly as obtained from the source or following a pretreatment to modify the character of the sample. The sample can be derived from any biological source, such as tissues, bodily fluids, extracts, or cell cultures, including cells (e.g. neuronal cells), cell lysates, and physiological fluids, such as, for example, whole blood, plasma, serum, saliva, ocular lens fluid, cerebral spinal fluid, sweat, urine, milk, ascites fluid, synovial fluid, peritoneal fluid and the like. Therefore, a biological sample may be blood, urine, saliva, a tissue biopsy, or autopsy material or material comprising neuronal cells. In an embodiment, the sample is serum. In a preferred embodiment the sample is from the CSF and taken by lumbar puncture. The sample can be obtained from animals, preferably mammals, most preferably humans. The sample can be treated prior to use, such as preparing plasma from blood, diluting viscous fluids, and the like. Methods of treatment can involve filtration, distillation, extraction, concentration, inactivation of interfering components, the addition of reagents, and the like. Proteins may be isolated from the samples and utilized in the methods of the invention.

Screening Assays and Methods

In one aspect the invention provides a method for modulating γ-secretase activity in-vitro in a sample or in-vivo in a subject comprising administering to said sample or subject a presenilin complex associated peptide. such as TMP21 or obvious chemical equivalent thereof. In one embodiment, the TMP21 peptide modulates, e.g. inhibits, γ-secretase activity but not ε-secretase activity. In one embodiment, the invention provides a use of TMP21 for inhibiting γ-secretase activity but not ε-secretase activity.

In one aspect the invention provides a method for decreasing Aβ production comprising inhibiting γ-secretase activity by administering to said sample TMP21 or obvious chemical equivalent thereof In one aspect TMP21 can be administered to a sample by administering a nucleotide sequence encoding TMP21 under conditions of expression of said nucleotide sequence encoding TMP21.

In another aspect the invention provides a method of identifying modulators of γ-secretase activity that are not modulators of ε-secretase activity comprising incubating γ-secretase or a biologically active source therefore with APP substrate under conditions wherein the secretase would cleave the APP to form Aβ, monitoring Aβ production in both the presence and absence (control) of a potential modulator, wherein a change in Aβ production as compared to the control is indicative of a modulator. In one aspect the method further comprises monitoring levels of ε-secretase activity, and selecting modulators that have no change in ε-secretase activity as compared to a control. In a further aspect, the method as noted above wherein the ε-secretase activity is monitored by monitoring levels of intracellular fragments of Notch and/or Cadherin (eg NICD or CICD) production. In another aspect, the method the modulator is an inhibitor of γ-secretase activity and has lower Aβ production levels as compared to a control. In another aspect, the potential modulator is first screened in a TMP21 binding assay and was determined to bind TMP21.

In another embodiment, the invention provides a method for screening for TMP21 modulators that selectively regulate γ secretase comprising: incubating APP with γ-secretase under conditions that would result in Aβ production, exposing said APP, gamma-secretase sample to a potential inhibitor of gamma secretase activity, monitoring the effect of said activity on Aβ production as compared to a control.

Compounds which modulate the biological activity of γ secretase may also be identified by comparing the pattern and level of expression of the protein in tissues and cells, in the presence, and in the absence of the compounds. In addition, compounds that modulate the biological activity of a γ secretase may be identified by assaying for modulation (i.e. inhibition or enhancement) of enzymatic activity.

The methods of the invention can be done through cell or cell free systems. The methods of the can be due by TMP21 in the The methods of the invention can be done using TMP21 in the assay and/or as part of the control. For instance, assays can be performed using TMP21 in the presence and absence of the potential modulator and results compared. In another embodiment, the assay can be done in the presence of TMP21 and a known inhibitor of TMP21, such as siRNA, and the effect of the presence of the potential modulator compared to that of the absence of the potential modulator on gamma-secretase and optionally e-secretase activity or indicators of same.

In one embodiment, levels of TMP21 are monitored directly or indirectly. For instance TMP21 levels can be monitored directly using RT-PCR, antibodies or other labeling agents of TMP21 known in the art, and through ELISA's, radiolabeling or other methods known to a person skilled in the art. Nucleotides encoding TAMP can also be monitored using techniques known in the art.

In one embodiment, TMP21 levels are monitored indirectly through indicators of TMP21 activity such as levels of gamma-secretase or Abeta.

Diagnostic and Pharmaceutical Uses

The invention further provides a method for preventing or treating a condition associated with γ-secretase activity but not ε-secretase activity comprising administering to a subject an effective amount of an inhibitor of γ-secretase activity but not ε-secretase activity, such as TMP21 or obvious chemical equivalent thereof. In one aspect the condition is an amyloid Aβ-related condition, such as Alzheimer's, cerebral amyloid angiopathy, and inclusion body myositis. Administering TMP21 to subject can include administering the peptide or precursor thereof per se or a nucleotide sequence encoding said peptide through gene therapy or cells expressing said peptide. In one embodiment, the invention provides a use of TMP21 or obvious chemical equivalent thereof in the prevention or treatment of a gamma-secretase related condition or an Abeta related condition.

The invention further provides a method for diagnosing a γ-secretase related condition comprising obtaining a biological sample from a subject that comprises presenilin complexes, determining TMP21 levels in said sample, comparing the TMP21 level with control levels from patients with known disease states, diagnosing the subject based on comparing TMP21 levels in said patient to the control levels and rendering a diagnosis based on said comparison with patients of known disease state. In one aspect the biological sample is measured in the CSF following lumbar puncture.

In one aspect, the TMP21 levels are determined directly, such as through assays that determine TMP21 expression levels (e.g. RT-PCR, PCR of TMP21 nucleotide coding sequences, Northern or Western blot analysis of TMP21, Elisa using an antibody to TMP21, radiolabelling or other methods known in the art).

In another aspect TMP21 levels are determined indirectly, by assessment of γ-secretase activity or Aβ production or other indicator of TMP21 levels.

In one aspect, the control levels are based on subjects with no γ-secretase related condition, wherein TMP21 levels that are lower than those of the control is indicative of a γ-secretase related condition. In one aspect, the γ-secretase related condition is selected from the group consisting of Alzheimer's, cerebral amyloid angiopathy, and inclusion body myositis. In one aspect the condition is Alzheimer's.

The invention further provides a method of monitoring the disease state of a subject with a γ-secretase related condition comprising monitoring levels of TMP21 activity in biologicial samples obtained from a subject over time, wherein a decrease in TMP21 levels over time is indicative of a worsening of or progression of the condition, while maintaining or increasing TMP21 levels over time is indicative of non-progression of the disease state. In one aspect, the method is used for monitoring disease progression wherein TMP21 is being used in the treatment of the condition.

[Insert Controls, Insert Cell Assays, Insert How TMP21, etc Measured. Insert Labelling of TMP21. can be Nucleic Acid Encoding TMP21]

Pharmaceutical Compositions

The invention also provides a pharmaceutical composition comprising TMP21 or obvious chemical equivalent thereof, such as pharmaceutically acceptable salt thereof, and a pharmaceutically acceptable carrier. The invention further provides for pharmaceutical compositions comprising regulators of γ secretase related activity but not ε-secretase activity, identified through the methods of the invention

The agents/compounds identified using the methods of the invention may be formulated into compositions for administration to individuals suffering from a γ secretase related condition disease or condition. Therefore, the present invention also relates to a composition comprising one or more of an agent/compound identified using a method of the invention, and a pharmaceutically acceptable carrier, excipient or diluent.

Thus, the agents/compounds identified using the methods of the invention may be formulated into compositions for administration to individuals suffering from a γ secretase related condition. Still further the invention provides the use of agent/compound identified using a method of the invention in the preparation of a medicament to treat individuals suffering from a γ secretase related condition disease or condition.

In an embodiment, the invention provides the use of an agent in the preparation of a medicament to modulate γ secretase activity but not ε-secretase activity.

An agent or compound herein can be administered to a subject either by themselves, or they can be formulated into pharmaceutical compositions for administration to subjects in a biologically compatible form suitable for administration in vivo. By “biologically compatible form suitable for administration in vivo” is meant a form of the agent/compound to be administered in which any toxic effects are outweighed by the therapeutic effects. The agents/compounds may be administered to living organisms including humans, and animals (e.g. dogs, cats, cows, sheep, horses, rabbits, and monkeys). Preferably the agents/compounds are administered to human and veterinary patients.

An agent/compound may be administered in a therapeutically active or effective amount. A “therapeutically active amount” or “therapeutically effective amount” or “effective amount” is defined as an amount of a substance, at dosages and for periods of time necessary to achieve the desired result. For example, a therapeutically active amount of an agent/compound may vary according to factors such as the disease state, age, sex, and weight of the individual, and the ability of the agent/compound to elicit a desired response in the individual. Dosage regime may be adjusted to provide the optimum therapeutic response. For example, several divided doses may be administered daily or the dose may be proportionally reduced as indicated by the exigencies of the therapeutic situation. A therapeutically active amount can be estimated initially either in cell culture assays e.g. of neruonal cells, or in animal models such as mice, rats, rabbits, dogs, or pigs. Animal models may be used to determine the appropriate concentration range and route of administration for administration to humans.

The active substance may be administered in a convenient manner by any of a number of routes including but not limited to oral, subcutaneous, intravenous, intraperitoneal, intranasal, enteral, topical, sublingual, intramuscular, intra-arterial, intramedullary, intrathecal, inhalation, transdermal, or rectal means. The active substance may also be administered to cells in ex vivo treatment protocols. Depending on the route of administration, the active substance may be coated in a material to protect the substance from the action of enzymes, acids and other natural conditions that may inactivate the substance.

The compositions described herein can be prepared by per se known methods for the preparation of pharmaceutically acceptable compositions which can be administered to subjects, such that an effective quantity of the active substance is combined in a mixture with a pharmaceutically acceptable vehicle. Suitable vehicles are described, for example, in Remington's Pharmaceutical Sciences (Remington's Pharmaceutical Sciences, Mack Publishing Company, Easton, Pa., USA 1985). On this basis, the compositions include, albeit not exclusively, solutions of the agents or compounds in association with one or more pharmaceutically acceptable vehicles or diluents, and contained in buffered solutions with a suitable pH and iso-osmotic with the physiological fluids.

An agent or compound can be in a composition which aids in delivery into the cytosol of a cell. The substance may be conjugated with a carrier moiety such as a liposome that is capable of delivering the substance into the cytosol of a cell (See for example Amselem et al., Chem. Phys. Lipids 64:219-237, 1993 which is incorporated by reference). Alternatively, an agent or compound may be modified to include specific transit peptides or fused to such transit peptides that are capable of delivering the substance into a cell. The agents or compounds can also be delivered directly into a cell by microinjection.

An agent or compound may be therapeutically administered by implanting into a subject, vectors or cells capable of producing the agent or compound. In one approach cells that secrete an agent or compound may be encapsulated into semipermeable membranes for implantation into a subject. The cells can be cells that have been engineered to express an agent or compound. It is preferred that the cell be of human origin.

A nucleic acid encoding an agent or compound may be used for therapeutic purposes. Viral gene delivery systems may be derived from retroviruses, adenoviruses, herpes or vaccinia viruses or from various bacterial plasmids for delivery of nucleic acid sequences to the target organ, tissue, or cells. Vectors that express the agent or compound can be constructed using techniques well known to those skilled in the art (see for example, Sambrook et al.). Non-viral methods can also be used to cause expression of an agent or compound in tissues or cells of a subject. Most non-viral methods of gene transfer rely on normal mechanisms used by mammalian cells for the uptake and transport of macromolecules. Examples of non-viral delivery methods include liposomal derived systems, poly-lysine conjugates, and artificial viral envelopes.

In viral delivery methods, vectors may be administered to a subject by injection, e.g. intravascularly or intramuscularly, by inhalation, or other parenteral modes. Non-viral delivery methods include administration of the nucleic acids using complexes with liposomes or by injection; a catheter or biolistics may also be used.

The activity of an agent, compound, or compositions of the invention may be confirmed in animal experimental model systems. The therapeutic efficacy and safety of an agent, compound, or composition can be determined by standard pharmaceutical procedures in cell cultures or animal models. Therapeutic efficacy and toxicity may be determined by standard pharmaceutical procedures in cell cultures or with experimental animals, such as by calculating the ED₅₀ (the dose therapeutically effective in 50% of the population) or LD₅₀ (the dose lethal to 50% of the population) statistics. The therapeutic index is the dose ratio of therapeutic to toxic effects and it can be expressed as the ED₅₀/LD₅₀ ratio. Pharmaceutical compositions which exhibit large therapeutic indices are preferred.

Antibodies that specifically bind a therapeutically active ingredient may be used to measure the amount of the therapeutic active ingredient in a sample taken from a patient for the purposes of monitoring the course of therapy.

The invention also contemplates a method for evaluating a γ secretase related condition or disease of a patient suspected of exhibiting a condition or disease involving a protein levels or a protein-protein interaction, such as TMP21 and γ secretase. For example, biological samples from patients suspected of exhibiting a disease or condition may be assayed for the presence of the interaction using a method of the invention. The development of the disease or condition is caused by an abnormal quantity of one or both proteins of the interaction, the assay should compare levels of the interaction in the biological sample to the range expected in normal tissue of the same type. Identification of differences may assist in the diagnosis, prognosis, or treatment of a disease or condition.

The present invention is described in the following Examples, which are set forth to aid in the understanding of the invention, and should not be construed to limit in any way the scope of the invention as defined in the claims which follow thereafter.

### EXAMPLES

Methods

Immunoaffinity Purification of Presenilin 1 Complex and Trypsinization

Membrane proteins were purified in parallel from wild-type and PS1^{-/-}/PS2^{-/-} blastocyst-derived cells extracted with buffer A (25 mM Hepes pH 7.4, 150 mM NaCl, 2 mM EDTA, protease inhibitor cocktail (Sigma)) containing 1% CHAPSO. After ultracentrifugation, solubilized membrane fractions were subjected to immunopurification of PS1 complexes with the use of Protein A-Sepharose that had been saturated and chemically cross-linked with an affinity-purified polyclonal rabbit antibody directed against the N terminus of PS-1. Captured proteins were pH-drop eluted, denatured in 6 M urea, reduced/alkylated and subjected to in-solution trypsinization. Proteolytic fragments were analyzed with two-dimensional liquid chromatography coupled to electrospray tandem mass spectrometry as described (26).

Immunoprecipitations, Co-Immunoprecipitations and Immunoblotting

Immunoprecipitations were performed in buffer A containing 1% Nonidet P-40 (27). Co-immunoprecipitations were performed in buffer A containing 1% digitonin or CHAPSO as the solubilizing detergent (3, 27, 31, 23). For Western blotting, proteins were separated on conventional Laemmli SDS-PAGE gels for standard protein samples or 16% tricine or 10% Bicine/Tris gels for the detection of Aβ. After immunoblotting on nitrocellulose, protein bands were detected by enhanced chemiluminescence (ECL; Amersham Biosciences). In some co-immunoprecipitation experiments, anti-rabbit or anti-mouse IgG (Fc) secondary antibodies (Pierce) were used for the detection of target proteins.

Immunocytochemistry

Immunocytochemistry was performed on HEK293 cells that had been grown on collagen-coated glass coverslips in Dulbecco's modified Eagle's medium (Invitrogen) supplemented with 20% fetal calf serum. Following 30 minutes fixation in ice-cold 4% formaldehyde in PBS cells were permeabilized by immersing coverslips for 30 minutes in PBS with 0.02% Triton X-100. Unsaturated binding sites were blocked by incubation for 2 hours at room temperature in blocking buffer (PBS supplemented with 5% normal goat serum pH 7.4). The cover slips were exposed overnight at 4° C. to primary antibodies (TMP21 antibody, NCT antibody and PS1 antibody), or organelle marker antibodies (Bip, βCOP and GM130). Following three washes in PBS, secondary labeling was done with Cy2-coupled anti-mouse (Jackson Laboratories; 1:800 dilution) and Cy3-coupled anti-rabbit (Jackson Laboratories; 1:800 dilution) antibodies for 2 hours at room temperature.

Subcellular Fractionation on Iodixanol Gradients

HEK293 cells; mouse blastocyst-derived cells from wild-type and from PS1^{-/-}, PS2^{-/-} mice; or brains dissected from wild-type mice, were homogenized with ice-cold Homogenization Buffer (130 mM KCl, 25 mM NaCl, 1 mM EGTA, protease inhibitor cocktail (Sigma), 25 mM Tris, pH 7.4) and extracts centrifuged first at 1,000×g for 10 minutes and subsequently at 3,000×g for 10 minutes. The resulting supernatants were layered on a step gradient consisting of 1 ml each of 30, 25, 20, 15, 12.5, 10, 7.5, 5, and 2.5% (v/v) iodixanol (Accurate) in Homogenization Buffer. After centrifugation at 27,000 rpm (SW40 rotor, Beckman) for 30 minutes, 11 fractions were collected from the top of the gradient. The fractions were analyzed for the presence of TMP21, components of γ-secretase and protein markers of subcellular organelles by Western blotting (31).

Glycerol Velocity Gradient

HEK293 cells were washed with ice-cold PBS, resuspended in 5 mM HEPES, pH 7.4, 1 mM EDTA, 0.25 M sucrose plus protease inhibitors, and homogenized, and the postnuclear supernatant was prepared as described previously (3, 6). Microsomal membranes were pelleted from the postnuclear supernatant by centrifugation at 100,000×g for 1 hour at 4° C. and solubilized in 1% CHAPSO, 50 mM Tris-HCl, pH 7.5, 2 mM EDTA, and 150 mM NaCl, plus protease inhibitors. The lysates were re-centrifuged at 100,000×g for 30 minutes, and the supernatants were used for glycerol gradient centrifugation fractionation experiments as described previously (3, 6). In brief, 1 ml of total protein extracts was applied to the top of an 11.0-ml 10-40% (w/v) linear glycerol gradient containing 25 mM HEPES, pH 7.2, 150 mM NaCl, and 0.5% CHAPSO. Gradients were centrifuged for 15 hours at 35,000 rpm and 4° C. using a Beckman SW41 rotor and were collected into 1.0-ml fractions from the top of the centrifugation tube.

Two-Dimensional Gel Electrophoresis of Presenilin 1 Complexes

Two-dimensional gel electrophoresis was performed as described previously (6).

The proteins in CHAPSO-solubilized membrane fractions were separated on a 5-13% Blue Native polyacrylamide gel, followed by a second dimension on a NuPAGE BisTris 4-12% precast 2-D gel (Invitrogen) for SDS-PAGE. Marker proteins used for BN-PAGE were thyroglobulin, 669 kDa; apoferritin, 443 kDa; β-amylase, 200 kDa; alcohol dehydrogenase 150 kDa, and carbonic anhydrase 29 kDa (Sigma) (6).

Cell Surface Biotinylation

The biotinylation was performed as previously described (32). In brief, HEK293 cells were washed three times with ice-cold PBS (pH 8.0; 1 mM MgCl2) and incubate with or without 1 mg/ml EZ-Link Sulfo-NHS-LC-biotin (Pierce) for 30 minutes at 4° C. The reaction was stopped by washing the cells once and then incubating for 15 minutes on ice with 20 mM glycine in PBS (pH 8.0; 1 mM MgCl2). The cells were collected and incubated in the lysis buffer (50 mM Tris, pH 7.4, 150 mM NaCl, 2 mM EDTA, 2 mM EGTA, 1% CHAPSO, Complete protease inhibitor cocktail; Roche) for 1 hour. The resulting lysates were affinity-purified with UltraLink Immobilized NeutrAvidin Plus (Pierce) overnight at 4° C. Bound proteins were eluted by boiling the beads for 5 minutes? in SDS-PAGE-sample buffer. The bound and unbound proteins were separated on 10-20% Tricine SDS-polyacrylamide gels.

TMP21 and p24a Complementary DNAs and Transfections

Untagged TMP21, TMP21 tagged at the C terminus with the Flag epitope, and untagged p24a were generated by cloning the respective cDNAs into pcDNA4 or pcDNA6 expression vectors (Invitrogen). These constructs were transiently or stably transfected (Lipofectamine 2000) into native HEK-293 cells, into a derivative HEK-293 cell line that stably expresses the Swedish APP mutant (APPswedish), or into blastocyst-derived mouse cells (28).

Notch Assays and Cadherin

Notch and cadherin cleavage assays were performed as described previously (27, 29).

Metabolic Labeling and Immunoprecipitation Procedure for Notch Assay: For pulse-chase experiments, expression of target genes was suppressed by transient transfection with siRNA oligos in HEK293 cells that stably express myc-tagged NotchΔE (25). Following a 20-minute metabolic labeling pulse with Trans-³⁵S-labeling reagent (ICN Pharmaceuticals), protein expression was chased for up to 2 hours. Cell lysates were subjected to immunoprecipitation with anti-myc antibody, as previously described (27).

Cadherin processing assay: Assays were performed as previously described (29). Briefly, cells were resuspended in 0.5 ml/35-mm dish of hypotonic buffer (10 mM MOPS, pH 7.0, 10 mM KCl) and homogenized on ice. A post-nuclear supernatant was prepared by centrifugation at 1000×g for 15 minutes at 4° C. Crude membranes were isolated from the post-nuclear supernatant by centrifugation at 16,000×g for 40 minutes at 4° C. The membranes were then resuspended in 25 μl of assay buffer (150 mM sodium citrate, pH 6.4, 1× Complete protease inhibitor cocktail, Roche), and incubated at 37° C. for 4 hours in the presence or absence of γ-secretase inhibitor (L-685,458, 1 μM). Samples were then analyzed by immunoblotting using C32 anti-N-cadherin and C36 anti-E-cadherin antibodies (BD Transduction Laboratories).

RNA Interference

siRNA-based knockdowns of target proteins were performed as described, in HEK-293 or blastocyst-derived cells (30). The oligonucleotide sequences are available in Supplementary Information.

TMP21 siRNA Oligos

Dharmacon RNA Technologies, siGENOMESMART pool reagent NM-006827.

(SEQ ID NO:4)

| | |
|---|---|
| hTmp21 A sense: | GCGGAUACCUGACCAACUCUU, |

(SEQ ID NO:5)

| | |
|---|---|
| anti-sense | 5′-PGAGUUGGUCAGGUAUCCGCUU; |

(SEQ ID NO:6)

| | |
|---|---|
| hTmp21 B sense | UCACAAGGACCUGCUAGUGUU, |

(SEQ ID NO:7)

| | |
|---|---|
| anti-sense | 5′-PCACUAGCAGGUCCUUGUGAUU; |

(SEQ ID NO:8)

| | |
|---|---|
| hTmp21 C sense | GCCAUAUUCUCUACUCCAAUU, |

(SEQ ID NO:9)

| | |
|---|---|
| anti-sense | 5′-PUUGGAGUAGAGAAUAUGGCUU; |

(SEQ ID NO:10)

| | |
|---|---|
| hTmp21 D sense | GAGCUGCGACGCCUAGAAGUU, |

(SEQ ID NO:11)

| | |
|---|---|
| anti-sense | 5′-PCUUCUAGGCGUCGCAGCUCUU. |

p24a siRNA Oligos:

5′-aaccggatgtccaccatgact-3′ (SEQ ID NO:12), 5′-acagagccatcaacgacaa-3′ (SEQ ID NO:13) (data not shown) and p24a pooled oligos (Dharmacon RNA Technologies, siGENOMESMART pool reagent, M-008074).

Negative control siRNA oligos: Dharmacon RNA Technologies, siGENOMESMART pool reagent D-001206).

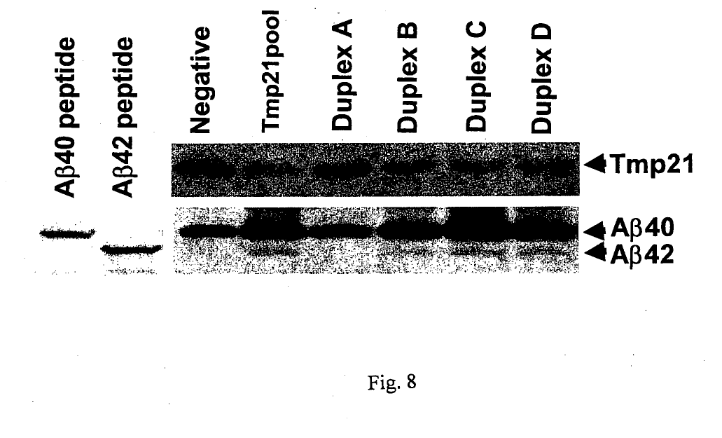

siRNA-based knockdowns of target proteins were carried out as described (30). Briefly, to inhibit expression of TMP21 or p24a by siRNA, HEK293 or blastocyst-derived cells were transiently transfected with a pool of all four TMP21 siRNA oligonucleotide pairs or p24a siRNA oligonucelotide pairs. As negative controls, we used a mixture of four pooled scrambled siRNAs and mock transfections without siRNA. The expression level of target proteins was monitored by Western blotting with polyclonal anti-TMP21 or anti-p24a antibodies. Each of the individual oligonucleotide pairs where independently shown to suppress the relevant target protein (FIG. 8).

Aβ Assays

Aβ40 and Aβ42 levels were measured by ELISA and immunoprecipitation-western blotting as described previously, with the use of 12-24-h-conditioned medium collected from native HEK-293 cells or from HEK-293 cells stably overexpressing APP (APPswedish or APPwt) (3,24). They were resuspended in Assay Buffer (10 mM KOAc, 1.5 mM MgCl₂, protease inhibitors (Roche), 75 mM sodium citrate, pH 6.4) and incubated at 0° C. or 37° C. for 2 or 4 hours (24). CHAPSO-solubilized membrane fractions from above cells were subjected to cell free γ-secretase assay as described below without addition of the C100-Flag substrate. Following adjustment to RIPA buffer, Aβ were captured by immmunoprecipitation with 6E10 antibody (Signet) and immunoblotted as described previously (24). ε-stubs were detected by Western blotting with anti-APP-CT antibody (Sigma).

Cell-free γ-secretase assays with endogenous APP were performed on microsome membranes from HEK-293 cells stably over expressing APPswedish with or without TMP21 RNAi treatments as described previously (24). ε-stubs were detected by western blotting with anti-APP-CT antibody (Sigma). Cell-free γ-secretase assay was performed as described (7, 13), with exogenous APP-C100 as the substrate was performed with recombinant APP-C100 peptides, wherein PS1 complex was isolated by immunoprecipitation from CHAPSO extracted membranes and combined with the recombinant C100 substrate and the generation of Aβ is determined by ELISA and AICD production is monitored by western blotting. More particularly, the peptides were generated from a prokaryotic expression vector encoding the C-terminal 100 amino acids (596-695) of human APP (695-residue isoform) followed by Flag and His₆ sequences (C100-Flag-His₆). This was generated by PCR and cloning into pQE60 (Qiagen). C100-Flag-His6 was expressed in E. coli BL21(DE3), purified as described (14) and was stored in the solution (20 mM Tris; pH 7.4, 500 mM NaCl and 10% glycerol) containing 0.5% NP-40 to stabilize the recombinant protein. CHAPSO-solubilized membranes or immunopurified PS1 complexes were incubated in reaction buffer without phosphatidylethanolamine or phosphatidylcholine for 6 hrs in the presence of <0.02% NP-40 (higher concentrations of NP-40 masked the increase in Aβ that paralleled TMP21 suppression). The generated Aβ was analyzed by immunoblotting or ELISA (Biosource).

Solubilized γ-secretase was prepared as described (7,13). Briefly, HEK293 cells were homogenized in HEPES Buffer (25 mM HEPES, pH 7.0, 150 mM NaCl, 5 mM MgCl₂, 5 mM CaCl₂, protease inhibitor cocktail). Postnuclear supernatants were centrifuged at 100,000 g for 1 hour to collect membrane pellets. These were washed with HEPES Buffer and subsequently resuspended in 1% CHAPSO/HEPES Buffer.Thus generated CHAPSO lysates were centrifuged at 100,000 g for 25 minutes to obtain supernatants containing solubilized γ-secretase preparations. Protein and detergent concentrations of solubilized γ-secretase preparations were adjusted to 0.25 mg/ml and 0.25% CHAPSO, respectively. Following addition of the C100-Flag substrate (˜0.5 μM) to 50 μl of the solubilized γ-secretase preparation the reaction mixture was incubated for 6 hrs at 37° C. and Aβ products detected by ELISA and Western blotting.

When γ-secretase assays were performed from immunoprecipitations, polyclonal anti-PS1-NTF antibody (A4) was added to 1% CHAPSO-solubilized membranes in buffer A (25 mM HEPES pH 7.4, 150 mM NaCl, 2 mM EDTA, with protease inhibitor cocktail (Sigma)). After incubation overnight at 4° C., beads were washed three times in buffer A containing 0.5% CHAPSO and once in HEPES buffer containing 25 mM HEPES, pH 7.0, 150 mM NaCl, 5 mM MgCl₂, 5 mM CaCl₂, protease inhibitor cocktail (Pierce), 0.25% CHAPSO. The samples were then resuspended in HEPES buffer containing 0.25% CHAPSO and 0.7 μM C100-Flag, and incubated for 6 h at 4° C. or 37° C. For complementation experiments, eluates from the TMP21-Flag immunoprecipitation (with anti-Flag M2-Agarose from mouse (Sigma)) from HEK-293 cells with or without TMP21-flag overexpression were added to the reaction system and incubated at 4° C. for 1 h followed by the addition of C100-Flag substrate (final concentration 0.7 μM) and incubation at 37° C. for 6 h. Western blotting served for both the detection of PS1 and the quantitative analysis of Aβ and ε-stubs after the elution of SDS sample buffer from the immunoprecipitation slurry.

### Example 1

### TMP21 Co-Precipitates with Other Presenilin Complex Components

In FIG. 1a, it can be seen that both endogenous and exogenous Flag-tagged TMP21 reciprocally co-precipitate known members of the presenilin complex from HEK-293 cells, SHSY-5Y cells and mouse brain (FIG. 1d). TMP21 also interacts with the p24 cargo protein p24a, but as a component of a separate complex. N.R.Sera IP, non-reactive serum immunoprecipitation control; mNCT, mature nicastrin; immNCT, immature nicastrin. Flag-TMP21, Flag-epitope-tagged TMP21, which was used in some experiments as indicated. In FIG. 1b, p24a, another member of the p24 cargo protein family, does not interact with any members of the presenilin complex but does co-precipitate TMP21. In FIG. 1c, TMP21 is expressed in many tissues including mouse brain (middle panel), neuron-like SHSY-5Y cells (top panel) and HEK293 cells (bottom panel). Both endogenous TMP21 (top two panels) and exogenous FLAG-tagged TMP21 (bottom panel) can be co-immunoprecipitated with endogenous PS1, NCT, aph-1, pen-2, but not with pre-immune sera. In FIG. 1d, TMP21 co-localizes with the other presenilin complex components in high molecular weight complexes (≧650 kDa) on glycerol velocity gradients. In FIG. 1e, on 2D-gel electrophoresis in wild type cells expressing endogenous proteins, TMP21 is distributed into a series of peaks, some of which overlap those of the mature, functional ˜660 kDa PS1 complex, the ˜440 kDa immature non-functional complex, and the ˜150 kDa nicastrin-aph-1 complex. About 25% of the TMP21 signal resides in the ˜660 kDa complex. In contrast, in both PS1/PS2 double knockout cells and in pen-2 knock-down cells, TMP21 is destabilized from the ˜660 kDa complex (<5% of signal intensity), and instead, TMP21 is predominantly localized with the ˜150 kDa nicastrin:aph-1 complex and in a 30 kDa complex. This suggests that TMP21 is likely added to the complex during its early maturation.

### Example 2

### Knock-Down of TMP21 Increases Aβ Production

In FIG. 2a, suppression of TMP21 by siRNA (left panel, middle column) causes increased γ-secretase activity and increased production of Aβ from whole cells, microsomal membranes, CHAPSO-solubilized membranes or immunopurified PS1 complexes. In contrast, suppression of another p24 cargo protein, p24a, by siRNA has no effect on Aβ production (right panel, right column). Control siRNA, scrambled nonsense siRNA oligonucleotides; mock, no siRNA oligonucleotides. APPs (secreted N-terminal ectodomain fragments of APP). In FIG. 2b, quantification of Aβ secretion by whole-cell (upper panel) or by cell-free (lower panel) γ-secretase assays after suppression of TMP21 or p24a by siRNA. Black bars, TMP21 siRNA; white bars, control scrambled siRNA; grey bars, p24a siRNA. Asterisk, P<0.01; two asterisks, P<0.001. Error bars show s.e.m. In FIG. 2c, for equivalent levels of p24a suppression (mediated by p24a siRNA only, by TMP21 siRNA or by both p24a siRNA and TMP21 siRNA) the suppression of p24a-associated TMP21 (by p24 siRNA only; lanes 3 and 4) had no effect on Aβ, whereas the subsequent additional suppression of the remaining free TMP21 (by p24a siRNA and TMP21 siRNA; lanes 7 and 8) causes an increase in Aβ. This increase in Aβ production in the double (p24a and TMP21) siRNA-suppressed cells (lanes 7 and 8) was equivalent to the increase in Aβ production induced by siRNA suppression of TMP21 alone (lanes 5 and 6). The figure is not directly intended to dissect the relative compartment sizes of free TMP21 and p24a-bound TMP21. However, by comparing the decrease in TMP21 signal between the p24a siRNA (lanes 3 and 4, which suppresses mostly p24a-bound TMP21) and the TMP21 siRNA (lanes 5 and 6, which suppresses both free TMP21 and p24a-bound TMP21), one can infer that in the presence of equivalent levels of p24a, the TMP21 siRNA induced an approximately 10-20% decrease in TMP21 signal intensity. This decrease represents the decrease in free TMP21. FIG. 2d, illustrates another role for TMP21 in addition to a p24 or 50 protein. In FIG. 2e, surface biotinylation of cells expressing endogenous TMP21 and endogenous presenilin complex components reveals that both nicastrin and TMP21 can be surfaced biotinylated, whereas intracellular proteins such as GM130 cannot be biotinylated (FIG. 2d).

### Example 3

### Complementation of TMP21-Deficient Presenilin Complexes (TMP21 siRNA) by Exogenous Immunopurified TMP21 (+TMP21-Flag) Reverts γ-Secretase Activity Towards Levels Observed in Wild-Type Control Complexes (Control siRNA)

Complementation of TMP21-deficient presenilin complexes (TMP21 siRNA) by exogenous immunopurified TMP1 (+TMP21-Flag) reverts γ-secretase activity towards levels observed in wild-type control complexes (Control siRNA) (FIG. 3a). In contrast, Aβ production remains elevated in TMP21-deficient complexes treated with anti-Flag immunoprecipitates lacking TMP21 (+control, no TMP21-Flag). Error bars show s.e.m. (FIG. 3b). In FIG. 3c, moderate over-expression of TMP21 has no discernible effect on Aβ production from whole cells or from cell-free γ-secretase assays using microsomal membranes or CHAPSO-solubilized membrane proteins. In FIG. 3d, TMP21 expression in SHSY-5Y cells was suppressed by RNAi. PS1 complexes were immuno-purified with anti-PS1 (A4) antibody from the CHAPSO-solubilized membrane fraction. γ-secretase activity as measured by Aβ generation was then assayed in vitro by incubation with C100-Flag at 37° C. for 6 hrs. As with HEK293 cells, TMP21 suppression caused increased Aβ production.

### Example 4

### The ε-Secretase Cleavage Site is not Affected by Knockdown of TMP21

FIG. 4a illustrates that TMP21 siRNA suppression does not affect either the cleavage of endogenous APP by ε-secretase to generate AICD by endogenous PS1 complexes in whole-cell assays or the ε-secretase-mediated cleavage of recombinant C100-APP (equivalent to the natural β-secretase-generated substrate) by immunopurified PS1 complexes in cell-free assays. In FIG. 4b, TMP21 siRNA has no effect on either the kinetics or the amount of cleavage of the Notch substrate (NotchΔE) by ε-secretase to generate NICD in whole cells. ko, knockout; wt, wild type. In FIG. 4c, TMP21 siRNA suppression has no effect on ε-cleavage of either E-cadherin (E-cad) or N-cadherin substrates (CAD/CTF1) to generate CAD/CTF2 C-terminal products. In contrast, this cleavage can be specifically inhibited by the γ/ε-secretase inhibitor L685,458.

### Example 5

### Conflict of Overexpression of p24a Activity

FIG. 5 illustrates that over-expression of p24a activity had no discernible effect on either γ-secretase (shown) or ε-secretase activity (not shown). Error bars represent mean±s.e.m.

### Example 6

### Presenilin-Dependent Endoproteolysis of TMP21

No evidence could be found for presenilin-dependent endoproteolysis of TMP21 (FIG. 6). No N-terminal soluble fragments were found in the media with the N-terminally-directed anti-TMP21 antibody (not shown). No fragments corresponding in size to γ-site cleaved C-terminal stubs could be found using a C-terminally tagged TMP21 construct either in wild-type HEK293 or murine blastocyst derived cells, or in the same cell types in which γ-/ε-secretase activity had been inhibited by: treated with 1 μM Compound E for 15 hrs (lane 1) or knockout of both PS1 and PS2 (lanes 4, 6).

### Example 7

### Effect of TMP21 siRAN Suppression

FIG. 7a illustrates that TMP21 siRNA suppression has no effect on the kinetics of APP trafficking or on the maturation of the N′O′-glycoslyation of APP on pulse-chase metabolic labeling studies.

FIG. 7b illustrates that TMP21 siRNA suppression had no affect on the total cellular levels of immature and maturely glycosylated nicastrin, APP, or β-secretase (BACE), but did dramatically reduced total cellular levels of TMP21 (left panel). Surface labeling experiments with biotin show that TMP21 suppression also had no effect on the abundance of cell surface nicastrin, APP, or BACE (right panel). Interestingly the abundance of TMP21 at the cell surface following 6 days of TMP21 suppression was also reduced (by ˜50% compared to mock siRNA or siRNA with nonsense oligos), this reduction was less than the reduction in total cellular TMP21. This supports the notion that TMP21 may be a stable component of PS1 complexes.

### Example 8

### Modulation of TMP21 Expression with TMP21 siRNA Oligonucleotide Pairs

Four independent TMP21 siRNA oligonucleotide pairs were designed (SEQ ID NOS: 4 and 5; 6 and 7; 8 and 9; 10 and 11). All four pairs reduced TMP21 expression, but to varying degrees. The resultant increase in Aβ production was proportionate to the reduction in TMP21 levels. For the majority of experiments, a pool of all four oligonucleotides was used (FIG. 8).

### Example 9

### Human γ-Secretase Reconstituted in Yeast can be Regulated by TMP21

Core γ-secretase enzyme activity of the mammalian CNS was reconstituted in S. cerevisiae by co-expression of human presenilin 1 (PS1), nicastrin (NCT), APH-1 and PEN-2 in accordance with the method described by Edbauer (12), with activity measured via endoproteolytic release of a Gal4 transcriptional activator from an APP_{C1-55} juxtamembrane region and trans activation of a β-galactosidase reporter gene. While a starting configuration performed slightly above the baseline of cells lacking γ-secretase subunits, non-fermentable carbon sources favoring oxidative metabolism increased output up to 100-fold and allowed profiling of the system. As in mammalian cells, activity was greater with wt PS1 than wt PS2, sensitive to missense mutations of APP substrate near the Aβ42 site, and was modulated by the membrane protein TMP21.

Besides the production of Aβ-related peptide fragments identified by mass spectroscopy, enzyme activity was measured via a transcriptional reporter assay wherein endoproteolysis of a membrane-tethered APP_{C1-55}/Gal4 fusion protein results in the release of a Gal4 transcriptional activator capable of driving a reporter gene (lacZ) with a colorimetric output. Effect of TMP21, Erv25p and Emp24p on reporter activity and PS1 processing

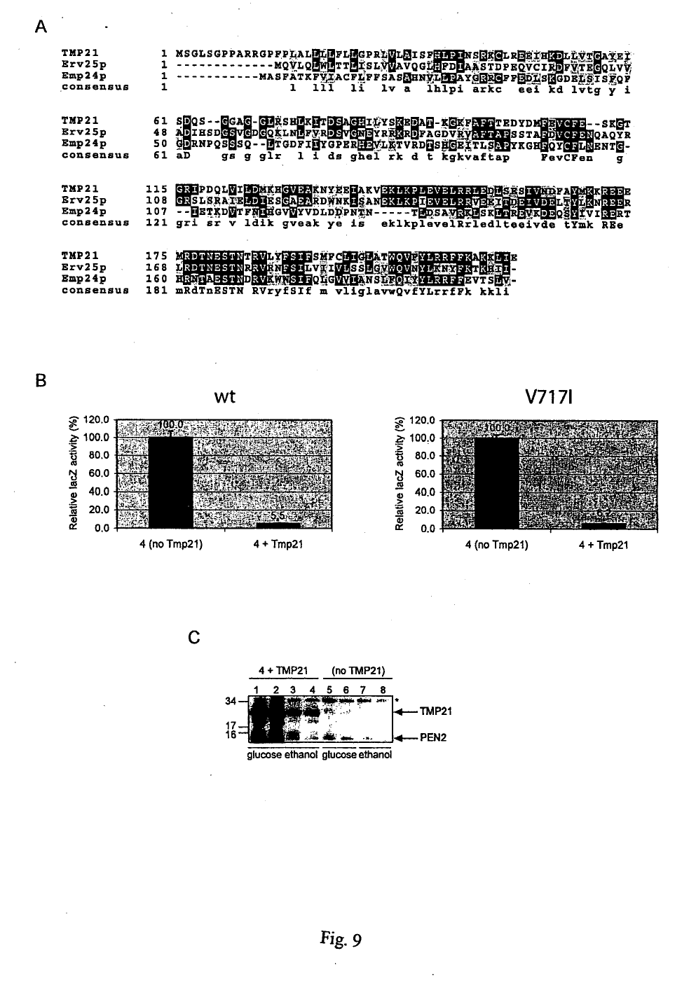

The inventors have demonstrated that TMP21, a member of the p24 cargo protein family first identified in S. cerevisiae, is a modulator of γ-secretase activity (33) (FIG. 9A). FIG. 9a Also illustrates sequence alignment of human TMP21 to the closest yeast homologue, Erv25p, with 58% sequence identity (FIG. 9A). Erv24p and Emp25p are components of COPII-coated vesicles and form a complex that is required for efficient transport from the endoplasmic reticulum (E.R.) to the Golgi (50).

When TMP21 expression is knocked down by siRNA, levels of Aβ40 and 42 increase 2-fold in both in vivo and in vitro mammalian systems. To assess the effects of human TMP21, the inventors exploited a pBEVY bi-cistronic expression vector incorporating a “nat1” gene. Here co-expression of TMP21 markedly reduced lacZ activity, irrespective of the APP genotype (i.e. wt or with a V717I mutation) of the reporter moiety (FIG. 9B), consistent with the suppressive activity seen in mammalian systems with TMP21. Expression of TMP21 was confirmed by Western analysis of transformants (FIG. 9C).

The APP-Gal4 based reporter system for γ-secretase bears important similarities to the mammalian prototype: (i) a dependence upon co-expression of γ-secretase sub-units (5, 6, 12, 13, 51), (ii) cleavage at Aβ40 and 42 sites demonstrated by mass spectroscopic analysis following mixing of prokaryotic APP C100 with yeast membrane preparations (12), (iii) an apparently superior performance of wt PS1 versus wt PS2 (52-55), an inhibitory effect of an APP C1-55 (V50F) mutation that decreases cleavage at Aβ42 but not at Aβ40, and (v) an inhibitory effect of a putative regulatory sub-unit TMP21 (33). In sum, these data strongly support γ-secretase mediated intramembraneous cleavage of a model TM1 protein substrate in the yeast system.

While the present invention has been described with reference to what is presently considered to be a preferred embodiment, it is to be understood that the invention is not limited to the disclosed embodiment. To the contrary, the invention is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety.

### REFERENCES

1. Sherrington, R. et al. Cloning of a gene bearing missense mutations in early-onset familial Alzheimer's disease. Nature 375, 754-760 (1995).
2. Rogaev, E. I. et al. Familial Alzheimer's disease in kindreds with missense mutations in a gene on chromosome 1 related to the Alzheimer's disease type 3 gene. Nature 376, 775-778 (1995).
3. Yu, G. et al. Nicastrin modulates presenilin-mediated notch/glp1 signal transduction and βAPP processing. Nature 407, 48-54 (2000).
4. Goutte, C., Tsunozaki, M., Hale, V. A. & Priess, J. R. APH-1 is a multipass membrane protein essential for the Notch signaling pathway in Caenorhabditis elegans embryos. Proc. Natl Acad. Sci. USA 99, 775-779 (2002).
5. Francis, R. et al. aph-1 and pen-2 are required for Notch pathway signaling, γ-secretase cleavage of βAPP, and presenilin protein accumulation. Dev. Cell 3, 85-97 (2002).
6. Gu, Y. et al. The presenilin proteins are components of multiple membrane-bound complexes which have different biological activities. J. Biol. Chem. 279, 31329-31336 (2004).
7. Li, Y.-M. et al. Presenilin 1 is linked with gamma-secretase activity in the detergent solubilized state. Proc. Natl Acad. Sci. USA 97, 6138-6143 (2000).
8. Evin, G. et al. Transition-state analogue γ-secretase inhibitors stabilize a 900 kDa presenilin/nicastrin complex. Biochemistry 44, 4332-4341 (2005).
9. De Strooper, B. et al. Deficiency of presenilin 1 inhibits the normal cleavage of amyloid precursor protein. Nature 391, 387-390 (1998).
10. De Strooper, B. et al. A presenilin-1-dependent γ-secretase-like protease mediates release of Notch intracellular domain. Nature 398, 518-522 (1999).
11. Marambaud, P. et al. A presenilin-1/γ-secretase cleavage releases the E-cadherin intracellular domain and regulates disassembly of adherens junctions. EMBO J. 21, 1948-1956 (2002).
12. Edbauer, D. et al. Reconstitution of γ-secretase activity. Nature Cell Biol. 5, 486-488 (2003).
13. Kimberly, W. T. et al. Gamma-secretase is a membrane protein complex comprised of presenilin, nicastrin, Aph-1, and Pen-2. Proc. Natl Acad. Sci. USA 100, 6382-6387 (2003).
14. Takasugi, N. et al. The role of presenilin cofactors in the γ-secretase complex. Nature 422, 438-441 (2003).
15. Blum, R. et al. Tmp21 and p24A, two type I proteins enriched in pancreatic microsomal membranes, are members of a protein family involved in vesicular trafficking. J. Biol. Chem. 271, 17183-17189 (1996).
16. Jenne, N., Frey, K., Brugger, B. & Wieland, F. T. Oligomeric state and stoichiometry of p24 proteins in the early secretory pathway. J. Biol. Chem. 277, 46504-46511 (2002).
17. Trower, M. K. et al. Conservation of synteny between the genome of the pufferfish (Fugu rubripes) and the region on human chromosome 14 (14q24.3) associated with familial Alzheimer disease (AD3 locus). Proc. Natl Acad. Sci. USA 93, 1366-1369 (1996).
18. Li, K. C., Liu, C. T., Sun, W., Yuan, S. & Yu, T. A system for enhancing genome-wide coexpression dynamics study. Proc. Natl Acad. Sci. USA 101, 15561-15566 (2004).
19. Wen, C. & Greenwald, I. p24 proteins and quality control of LIN-12 and GLP-1 trafficking in Caenorhabditis elegans. J. Cell Biol. 145, 1165-1175 (1999).
20. Luo, J. L., Kamata, H. & Karin, M. IKK/NF-κB signaling: balancing life and death—a new approach to cancer therapy. J. Clin. Invest. 115, 2625-2632 (2005).
21. Herranz, H., Stamataki, E., Feiguin, F. & Milan, M. Self-refinement of Notch activity through the transmembrane protein Crumbs: modulation of γ-secretase activity. EMBO Rep. (in the press).
22. Yu, C. et al. Characterization of a presenilin-mediated amyloid precursor protein carboxyl-terminal fragment γ. Evidence for distinct mechanisms involved in γ-secretase processing of the APP and Notch1 transmembrane domains. J. Biol. Chem. 276, 43756-43760 (2001).
23. Kopan, R. & Ilagan, M. X. Gamma-secretase: proteasome of the membrane? Nature Rev. Mol. Cell Biol. 5, 499-504 (2004).
24. Chen, F. et al. Presenilin 1 mutations activate γ 42-secretase but reciprocally inhibit ε-secretase cleavage of amyloid precursor protein (APP) and S3-cleavage of notch. J. Biol. Chem. 277, 36521-36526 (2002).
25. Petit, A. et al. New protease inhibitors prevent γ-secretase-mediated production of Aβ40/42 without affecting Notch cleavage. Nature Cell Biol. 3, 507-511 (2001).
26. Schmitt-Ulms, G. et al. Time-controlled transcardiac perfusion cross-linking for the study of protein interactions in complex tissues. Nature Biotechnol. 22, 724-731 (2004).
27. Chen, F. et al. Nicastrin binds to membrane-tethered Notch. Nature Cell Biol. 3, 751-754 (2001).
28. Donoviel, D. et al. Mice lacking both presenilin genes exhibit early embryonic patterning defects. Genes Dev. 13, 2801-2810 (1999).
29. Marambaud, P. et al. A CBP binding transcriptional repressor produced by the PS1/ε-cleavage of N-cadherin is inhibited by PS1 FAD mutations. Cell 114, 635-645 (2003).
30. Hasegawa, H. et al. Both the sequence and length of the C terminus of PEN-2 are critical for intermolecular interactions and function of presenilin complexes. J. Biol. Chem. 279, 46455-46463 (2004).
31. Chen, F. et al. Proteolytic derivative of Amyloid Precursor Protein accumulate in restricted and unpredicted intracellular compartments in the absence of functional presenilin 1 expression. J. Biol. Chem. 275, 36794-36802 (2000).
32. Chen, F. et al. Presenilin 1 and presenilin 2 have differential effects on the stability and maturation of nicastrin in Mammalian brain. J Biol Chem 278, 19974-9 (2003).
33. Chen, F. et al. TMP21 is a presenilin complex component that modulates gamma-secretase but not epsilon-secretase activity. Nature In press (2006) Nature 440, 1208-12.
34. Mattson, M. P. Pathways towards and away from Alzheimer's disease. Nature 430, 631-9 (2004).
35. Citron, M. et al. Mutant presenilins of Alzheimer's Disease increase production of 42 residue amyloid B-protein in both transfected cells and transgenic mice. Nature Med. 3, 67-72 (1997).
36. Katayama, T. et al. Presenilin-1 mutations downregulate the signalling pathway of the unfolded-protein response, Nature Cell Biol. 1, 479-485 (1999).
37. Pardossi-Piquard, R. et al. Presenilin-dependent transcriptional control of the Abeta-degrading enzyme neprilysin by intracellular domains of betaAPP and APLP. Neuron 46, 541-54 (2005).
38. Barr, F. A., Preisinger, C., Kopajtich, R. & Korner, R. Golgi matrix proteins interact with p24 cargo receptors and aid their efficient retention in the Golgi apparatus. J Cell Biol 155, 885-91 (2001).
39. Blum, R. et al. Intracellular localization and in vivo trafficking of p24A and p23. J Cell Sci 112 (Pt 4), 537-48 (1999).
40. Katzman, R. & Kawas, C. in Alzheimer Disease (eds. Terry, R. D., Katzman, R. & Bick, K. L.) 105-122 (Raven Press, New York, 1994).
41. Rensink, A. A., de Waal, R. M., Kremer, B. & Verbeek, M. M. Pathogenesis of cerebral amyloid angiopathy. Brain Res Brain Res Rev 43, 207-23 (2003).
42. Engel, W. K. & Askanas, V. Inclusion-body myositis: clinical, diagnostic, and pathologic aspects. Neurology 66, S20-9 (2006).
43. Rozmahel, R. et al. Alleles at the Nicastrin locus modify presenilin 1-deficiency phenotype. Proc Natl Acad Sci USA 99, 14452-7. (2002).
44. Saura, C. A. et al. Loss of presenilin function causes impairments of memory and synaptic plasticity followed by age-dependent neurodegeneration. Neuron 42, 23-36 (2004).
45. Tomita, T. & Iwatsubo, T. The inhibition of gamma-secretase as a therapeutic approach to Alzheimer's disease. Drug News Perspect 17, 321-5 (2004).
46. Imbimbo, B. P. The potential role of non-steroidal anti-inflammatory drugs in treating Alzheimer's disease. Expert Opin Investig Drugs 13, 1469-81 (2004).
47. Tabet, N. & Feldman, H. Indomethacin for the treatment of Alzheimer's disease patients. Cochrane Database Syst Rev, CD003673 (2002).
48. Citron, M. Beta-secretase inhibition for the treatment of Alzheimer's disease—promise and challenge. Trends Pharmacol Sci 25, 92-7 (2004).
49. McLaurin, J. et al. Therapeutically effective antibodies against amyloid-beta peptide target amyloid-beta residues 4-10 and inhibit cytotoxicity and fibrillogenesis. Nat Med 8, 1263-9. (2002).
50. Belden, W. J. & Barlowe, C. (1996) J Biol Chem 271, 26939-46.
51. Marlow, L., Canet, R. M., Haugabook, S. J., Hardy, J. A., Lahiri, D. K. & Sambamurti, K. (2003) Biochem Biophys Res Commun 305, 502-9.
52. Mastrangelo, P., Mathews, P. M., Chishti, M. A., Schmidt, S. D., Gu, Y., Yang, J., Mazzella, M. J., Coomaraswamy, J., Horne, P., Strome, B., Pelly, H., Levesque, G., Ebeling, C., Jiang, Y., Nixon, R. A., Rozmahel, R., Fraser, P. E., St George-Hyslop, P., Carlson, G. A. & Westaway, D. (2005) Proc Natl Acad Sci USA 102, 8972-7.
53. Qi, Y., Morishima-Kawashima, M., Sato, T., Mitsumori, R. & Ihara, Y. (2003) Biochemistry 42, 1042-52.
54. Tomita, T., Tokuhiro, S., Hashimoto, T., Aiba, K., Saido, T. C., Maruyama, K. & Iwatsubo, T. (1998) J Biol Chem 273, 21153-60.
55. Oyama, F., Sawamura, N., Kobayashi, K., Morishima-Kawashima, M., Kuramochi, T., Ito, M., Tomita, T., Maruyama, K., Saido, T. C., Iwatsubo, T., Capell, A., Walter, J., Grunberg, J., Ueyama, Y., Haass, C. & Ihara, Y. (1998) J Neurochem 71, 313-22.

### SEQUENCE LISTING

TMP21 (accession number Q9D1D4) based on three unique tryptic peptides (LKPLEVELR (SEQ ID NO:1), IPDQLVILDMK (SEQ ID NO:2) and RLEDLSESIVNDFAYMK (SEQ ID NO:3)) covering 17.5% of the full protein length. TMP21, a 219-amino-acid type 1 transmembrane protein, is a member of the p24 cargo-protein family (15)

TMP21 siRNA Oligos

Dharmacon RNA Technologies, siGENOMESMART pool reagent NM-006827.

(SEQ ID NO:4)

| | |
|---|---|
| hTmp21 A sense: | GCGGAUACCUGACCAACUCUU, |

(SEQ ID NO:5)

| | |
|---|---|
| anti-sense | 5′-PGAGUUGGUCAGGUAUCCGCUU; |

(SEQ ID NO:6)

| | |
|---|---|
| hTmp21 B sense | UCACAAGGACCUGCUAGUGUU, |

(SEQ ID NO:7)

| | |
|---|---|
| anti-sense | 5′-PCACUAGCAGGUCCUUGUGAUU; |

(SEQ ID NO:8)

| | |
|---|---|
| hTmp21 C sense | GCCAUAUUCUCUACUCCAAUU, |

(SEQ ID NO:9)

| | |
|---|---|
| anti-sense | 5′-PUUGGAGUAGAGAAUAUGGCUU; |

(SEQ ID NO:10)

| | |
|---|---|
| hTmp21 D sense | GAGCUGCGACGCCUAGAAGUU, |

(SEQ ID NO:11)

| | |
|---|---|
| anti-sense | 5′-PCUUCUAGGCGUCGCAGCUCUU. |

p24a siRNA Oligos:

5′-aaccggatgtccaccatgact-3′ (SEQ ID NO:12), 5′-acagagccatcaacgacaa-3′ (SEQ ID NO:13) (data not shown) and p24a pooled oligos (Dharmacon RNA Technologies, siGENOMESMART pool reagent, M-008074).

1. A method for modulating γ-secretase activity in-vitro sample or in-vivo in a subject comprising administering to said sample or subject TMP21 or obvious chemical equivalent thereof.

2. The method of claim claim wherein TMP21 is a presenilin complex associated peptide.

3. The method of claim 1 wherein the TMP21 or obvious chemical equivalent thereof modulates γ-secretase activity but not ε-secretase activity.

4. The method of claim 3 wherein the presenilin-complex associated peptide inhibits γ-secretase activity but not ε-secretase activity.

5. The method of claim 4 for decreasing Aβ production.

6. A method for preventing or treating a condition associated with γ-secretase activity but not ε-secretase activity comprising administering to a subject an effective amount of TMP21 or obvious chemical equivalent thereof.

7. The method of claim 6 wherein TMP21 or obvious chemical equivalent thereof inhibits γ-secretase activity but not ε-secretase activity.

8. The method of claim 7 wherein the condition is an amyloid Aβ-related condition.

9. The method of claim 8 wherein the amyloid Aβ-related condition is selected from the group consisting of: Alzheimer's, cerebral amyloid angiopathy, and inclusion body myositis.

10. The method of claim 9, wherein the condition is Alzheimer's.

11. A method for diagnosing a γ-secretase related condition comprising obtaining a biological sample from a subject that comprises presenilin complexes, determining TMP21 levels in said sample, comparing the TMP21 level with control levels from patients with known disease states, diagnosing the subject based on comparing TMP21 levels in said patient to the control levels and rendering a diagnosis based on said comparison with patients of known disease state.

12. The method of claim 11, wherein the TMP21 levels are determined directly.

13. The method of claim 12, wherein the TMP21 levels are determined by assessing levels of nucleotide sequence encoding TMP21.

14. The method of claim 12 wherein TMP21 levels are determined through binding studies.

15. The method of claim 14, wherein TMP21 levels are determined through use of an antibody that binds TMP21.

16. The method of claim 11 wherein the TMP21 levels are determined indirectly, by assessment of γ-secretase activity.

17. The method of claim 13, wherein γ-secretase activity is determined by Aβ production.

18. The method of claim 11, wherein the control levels are based on subjects with no γ-secretase related condition and TMP21 levels that are lower than those of the control is indicative of a γ-secretase related condition.

19. The method of claim 18, wherein the γ-secretase related condition is selected from the group consisting of Alzheimer's, cerebral amyloid angiopathy, and inclusion body myositis.

20. The method of claim 19, wherein the condition is Alzheimer's.

21. A method of monitoring the disease state of a subject with a γ-secretase related condition comprising monitoring levels of TMP21 activity in biological samples obtained from a subject over time, wherein a decrease in TMP21 levels over time is indicative of a worsening of or progression of the condition, while maintaining or increasing TMP21 levels over time is indicative of non-progression of the disease state.

22. The method of claim 21 for monitoring disease progression wherein TMP21 is being used in the treatment of the condition.

23. A method of identifying modulators of γ-secretase activity that are not modulators of ε-secretase activity comprising incubating γ-secretase or a biologically active source therefore with APP substrate under conditions wherein the secretase would cleave the APP to form Aβ, monitoring Aβ production in both the presence and absence (control) of a potential modulator, wherein a change in Aβ production as compared to the control is indicative of a modulator.

24. The method of claim 20, further comprising monitoring levels ε-secretase activity, and selecting modulators that have no change in ε-secretase activity as compared to a control.

25. The method of claim 24, wherein the ε-secretase activity is monitored by monitoring levels of intracellular fragments of Notch and/or Cadherin.

26. The method of claim 24, wherein the modulator is an inhibitor of γ-secretase activity and has lower Aβ production levels as compared to a control.

27. The method of claim 23, wherein the potential modulator is first screened in a TMP21 binding assay and was determined to bind TMP21.

28. The method of claim 23, wherein the control is the presence of TMP21 but no potential modulator and/or the presence of TMP21 plus the potential modulator, and/or the present of TMP21 and a known modulator of TMP21.

29. A method for screening for TMP21 modulators that selectively regulate γ secretase comprising: incubating APP with γ-secretase under conditions that would result in Aβ production, exposing said APP, gamma-secretase sample to a potential inhibitor of gamma secretase activity, monitoring the effect of said activity on Aβ production as compared to a control.

30. The method of claim 28, wherein the method is done in the presence and absence of TMP21 and any change in TMP21 activity in the presence of the potential modulator as compared to no potential modulator is indicative that the potential modulator is a modulator of TMP21.

31. The method of claim 29, wherein the potential modulator is first screened in a TMP21 binding assay and was determined to bind TMP21.

32. A pharmaceutical composition comprising TMP21, a pharmaceutically acceptable salt thereof or obvious chemical equivalent thereof and a pharmaceutically acceptable carrier.


SEQUENCE LISTING (13 sequences)

SEQ ID NO: 1 - protein, 9 aa, Mus musculus
  MISC_FEATURE: Tryptic peptide derived from TMP21
LKPLEVELR

SEQ ID NO: 2 - protein, 11 aa, Mus musculus
  MISC_FEATURE: Tryptic peptide derived from TMP21
IPDQLVILDMK

SEQ ID NO: 3 - protein, 17 aa, Mus musculus
  MISC_FEATURE: Tryptic peptide derived from TMP21
RLEDLSESIVNDFAYMK

SEQ ID NO: 4 - RNA, 21 nt, Artificial Sequence
  TMP21 siRNA sense strand
gcggauaccugaccaacucuu

SEQ ID NO: 5 - RNA, 21 nt, Artificial Sequence
  TMP21 siRNA antisense strand with 5′-phosphate terminus
gaguuggucagguauccgcuu

SEQ ID NO: 6 - RNA, 21 nt, Artificial Sequence
  TMP21 siRNA sense strand
ucacaaggaccugcuaguguu

SEQ ID NO: 7 - RNA, 21 nt, Artificial Sequence
  TMP21 siRNA antisense strand with 5′-phosphate terminus
cacuagcagguccuugugauu

SEQ ID NO: 8 - RNA, 21 nt, Artificial Sequence
  TMP21 siRNA sense strand
gccauauucucuacuccaauu

SEQ ID NO: 9 - RNA, 21 nt, Artificial Sequence
  TMP21 siRNA antisense strand with 5′-phosphate terminus
uuggaguagagaauauggcuu

SEQ ID NO: 10 - RNA, 21 nt, Artificial Sequence
  TMP21 siRNA sense strand
gagcugcgacgccuagaaguu

SEQ ID NO: 11 - RNA, 21 nt, Artificial Sequence
  TMP21 siRNA antisense strand with 5′-phosphate terminus
cuucuaggcgucgcagcucuu

SEQ ID NO: 12 - DNA, 21 bp, Artificial Sequence
  Target sequence of p24a siRNA oligonucleotide
aaccggatgtccaccatgact

SEQ ID NO: 13 - DNA, 19 bp, Artificial Sequence
  Target sequence of p24a siRNA oligonucleotide
acagagccatcaacgacaa